(19) United States
(12) Patent Application Publication
(10) Pub. No.: US 2015/0307532 A1
(43) Pub. Date: Oct. 29, 2015

(54) THIENOPYRIMIDINE INHIBITORS OF FARNESYL AND/OR GERANYLGERANYL PYROPHOSPHATE SYNTHASE

(71) Applicant: THE ROYAL INSTITUTION FOR THE ADVANCEMENT OF LEARNING/MCGILL UNIVERSITY, Montréal (CA)

(72) Inventors: Youla S. TSANTRIZOS, Montreal (CA); Judes POIRIER, Verdun (CA); Michael SEBAG, Montreal (CA); Albert BERGHUIS, Kirkland (CA); Jaeok PARK, Montreal (CA); Joris WIM DE SCHUTTER, Wilrujk (BE)

(73) Assignee: THE ROYAL INSTITUTION FOR THE ADVANCEMENT OF LEARNING/MCGILL UNIVERSITY, Montréal, QC (CA)

(21) Appl. No.: 14/646,304

(22) PCT Filed: Nov. 19, 2013
(86) PCT No.: PCT/CA2013/050884

§ 371 Date: May 20, 2015

Related U.S. Application Data
(60) Provisional application No. 61/728,489, filed on Nov. 20, 2012.

(51) Int. Cl. C07F 9/6561; C07D 495/04

(52) U.S. Cl. CPC C07F 9/6561; C07D 495/04

(57) ABSTRACT

The present invention relates to novel compounds, compositions containing same and methods for inhibiting human farnesyl pyrophosphate synthase or for the treatment or prevention of disease conditions using said compounds;

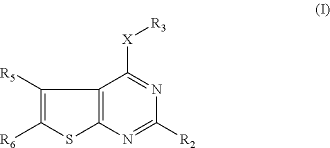

### CROSS REFERENCE TO RELATED APPLICATIONS

The present application is a national stage application of PCT/CA2013/050884 filed on Nov. 19, 2013, which claims priority on U.S. Provisional Patent Application No. 61/728,489 filed on Nov. 20, 2012. The entire contents of each of PCT/CA2013/050884 and U.S. Provisional Patent Application No. 61/728,489 are hereby incorporated herein by reference in their entirety.

### FIELD OF THE DISCLOSURE

The present invention relates to novel compounds, compositions containing same and methods for inhibiting the human farnesyl pyrophosphate synthase (hFPPS) and directly or indirectly the human geranylgeranyl pyrophosphate synthase (hGGPPS) for the treatment or prevention of disease conditions associated with overexpression of these enzymes and intracellular accumulation of their corresponding metabolites farnesyl pyrophosphate (FPP) and/or geranylgeranyl pyrophosphate (GGPP), respectively.

### BACKGROUND OF THE DISCLOSURE

The human farnesyl pyrophosphate synthase (hFPPS) enzyme is responsible for the catalytic elongation of dimethylallyl pyrophosphate (DMAPP) to geranyl pyrophosphate (GPP) and then to farnesyl pyrophosphate (FPP) via the successive condensation of two isopentenyl pyrophosphate (IPP) units (Scheme 1). Furthermore, farnesyl pyrophosphate (FPP) is the key metabolic precursor for the biosynthesis of geranylgeranyl pyrophosphate (GGPP), which is catalyzed by geranylgeranyl pyrophosphate synthase (GGPPS). Post-translational prenylation with FPP or GGPP of various proteins is crucial to their biological role. Consequently, inhibition of FPPS or GGPPS would result in decreased levels of both FPP and GGPP or only GGPP in a mammalian host, including a human host. Hence the human FPPS and GGPPS are recognized as important drug targets. It is anticipated that new FPPS or GGPPS inhibitors would have pleiotropic therapeutic effects, including in the treatment of bone diseases, in oncology, the treatment of elevated levels of cholesterol, prevention or treatment of neurodegenerative diseases (such as Alzheimer's), the treatment of infections, and any other disease state that is mediated by elevated levels of FPP or GGPP biosynthesis.

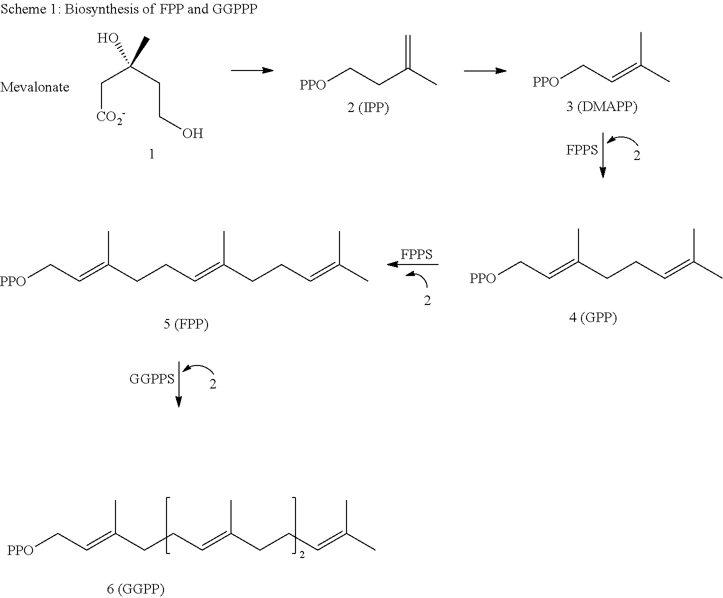

Farnesylation or geranylgeranylarion of proteins confers membrane localization, promotes specific protein-protein interactions and is believed to play a critical role in intracellular trafficking and signal transduction (see for example Nguyen U. T. T. et al. Nat. Chem. Biol. 2009, 5, 227-235). Addition of the FPP or GGPP lipidic moiety to the GTP-binding proteins (e.g. Ras, Rho, Rac, Rap) is also required in order to regulate the proliferation, invasive properties, and pro-angiogenic activity in human cancers (see for example Berndt, N.; Hamilton, A. D.; Sebti, S. M. Nature Rev. 2011, 11, 775-791; Caraglia, M. et al. Endocrine-Related Cancer 2006, 13, 7-26; Zhang, Y. et al. J. Am. Chem. Soc. 2009, 131, 5153-5162; Chapman, M. A. et al, Nature 2011, 471, 467-472.

The role of hFPPS in protein prenylation in osteoclasts is known (see for example Dunford, J. E. et al. J. Pharmacol. Exp. Ther. 2001, 296, 235-242; Marma, M. S. et al. J. Med. Chem. 2007, 50, 5967-5975 and 7. Dunford, J. E. et al. J. Med. Chem. 2008, 51, 2187-2195) and nitrogen-containing bisphosphonate (N-BP) inhibitors of hFPPS are commonly used in the treatment of osteoporosis, tumor-induced hypercalcemia, Paget's disease and osteolytic metastases (see Caraglia, M. et al, supra).

Inhibitors of hFPPS have also been reported to stimulate the immune system by indirectly activating Vγ2\762 T cells (also known as Vγ9\762 T cells), thus mediating antitumor and antimicrobial effects, more specifically broad-spectrum antiviral and antibacterial effects (see for example Sanders, J. M. et al. J. Med. Chem. 2004, 47, 375-384; Zhang, Y. et al. J. Med. Chem. 2007, 50, 6067-6079; Morita, C. T. et al. Immunological Reviews 2007, 215, 59-76; Breccia, P. et al. J. Med. Chem. 2009, 52, 3716-3722 and Li, J. et al. J. Immunol. 2009, 182, 8118-8124.

The antitumor effects of bisphosphonates inhibiting hFPPS (and/or its related enzyme hGGPPS) have been implicated in a variety of cancers (see Caraglia, M. et al, supra), including colorectal (see Notarnicola, M. et al. Oncology 2004, 67, 351-358), prostate, melanoma (see Laggner, U. et al. Clin. Immunol. 2009, 131, 367-373), breast (see for example Coleman, R. E. Eur. J. Cancer 2009, 45, 1909-1915), ovarian, brain (see Ellis, C. A. et al. Proc. Natl. Acad. Sci. USA 2002, 99, 9876-9881) and multiple myeloma (cancers. Nitrogen-containing bisphosphonate (N-BP) inhibitors of the human FPPS, such as zoledronic acid,) are disease modifying agents that improve survival in patients with multiple myeloma (MM) via mechanisms that are unrelated to their skeletal effects (see Morgan, G. J. et al. Lancet 2010, 376, 1989).

Inhibitors of FPPS may also be used for lowering cholesterol or treating infectious diseases caused by microorganisms (e.g. Staphylococcus aureaus) and protozoan parasites, such as the groups of Leishmania, Plasmodium, Trypanosoma, Toxoplasma, Cryptosporidium and others, by directly inhibiting the analogous FPPS enzyme of these organisms.

Recent literature also suggests that over-expression of hFPPS and hGGPPS is associated with neurodegeneration in the human Alzheimer's brain (see for example: Eckert, G. P.; Hooff, G. P.; Strandjord, D. M.; Igbavboa, U.; Volmer, D. A.; Müllner, W. E.; Wood, W. G. Neurobiol. Disease 2009, 35, 251-257 and Hooff, G. P.; Wood, W. G.; Müllner, W. E.; Eckert, G. P. Biochim. Biophys. Acta 2010, 1801, 896-905.). Accumulation of the phospho-Tau protein is strongly implicated in neuronal damage and the progression of diseases associated with dementia such as the Alzheimer's disease (see for example: Gong, C.-X.; Grundke-Iqbal, I.; Iqbal, K. Drug Aging 2010, 27, 351-365). Phospho-Tau levels can be modulated though the prenylation pathway from FPP→GGPP→RhoA→Cdc42→GSK3-β kinase→phospho-Tau protein. The use of statins (which indirectly down-regulate the biosynthesis of FPP and GGPP) in elderly subjects with normal cognitive functions is known to lead (over the course of several years) to a marked reduction of neurofibrillary tangle accumulation in the brain (detected at autopsy), as compared to non-users of statins. The potential benefits of statins in the treatment of Alzheimer's are currently under clinical investigation (see for example: Rebollo, A.; Pou, J.; Alegret, M. Aging Health 2008, 4, 171-180 and Mans, R. A.; McMahon, L. L. Li, L. Neuroscience 2012, 202, 1-9).

Therefore, the aim of the present invention is to provide novel hFPPS and/or hGGPPS inhibitors and methods for treating hFPPS-dependent or hGGPPS-dependent disorders with advantageous biopharmaceutical properties as compared to the current drugs that target the human FPPS.

Currently the only inhibitors of hFPPS that are approved drugs are the bisphosphonates, more importantly the nitrogen-containing bisphosphonates (N-BPs), for example, zoledronic acid and risedronic acid. Recently, non-bisphosphonate exploratory compounds, such as the examples shown below (compounds 1 and 2), are also reported to inhibit the human FPPS (see R. Amstutz et al. WO 2009/106586; W. Jahnke et al. Nature Chem. Biol. 2010, 6, 660-666).

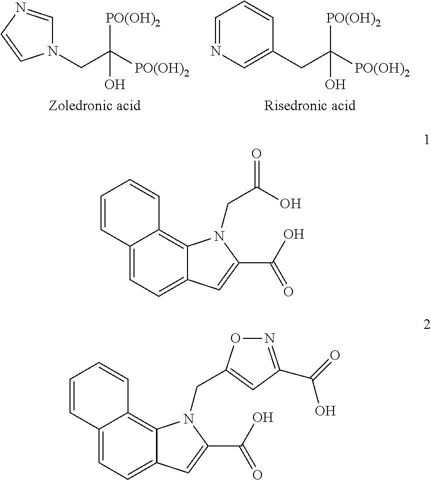

### SUMMARY

In an aspect of the disclosure, there is provided a compound of formula I:

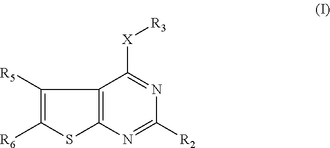

or a pharmaceutically acceptable salt or solvate thereof, wherein

### X═O, NR4, or CR4R4;

R2 is selected from H, C1-6alkyl, C3-6 cycloalkyl, C6-10aryl, 3-10 membered heterocycle, —CONHR7, —SO₂NHR7;
R3 is selected from CH[PO(OH)₂]₂; CH₂PO(OH)₂; CHR7PO(OH)₂; CH(CO₂H)₂; CH(SO₂NHR7)PO(OH)₂; CR8R9-SO₂NR7(PO(OH)₂), COCO₂H; CR8(PO(OH)₂)₂, CR8R9CO₂H; CR8R9PO(OH)₂, CR8R9COR10 or C1-6alkyl;
R4 are each independently H, C1-6alkyl, aryl or 3-10 membered heterocycle;
R5 and R6 are independently selected from H, C1-6alkyl, optionally substituted C3-6 cycloalkyl, optionally substituted C6-10aryl, optionally substituted 3-10 membered heterocycle, CH₂OH, CO₂H, CH₂CO₂H, (CH₂)ₙPO(OH)₂, (CH₂)ₙ—SO₂NR7(PO(OH)₂), (CH₂)ₙSO₂NR7R8, NR7R8, NH(CH₂), PO(OH)₂, NO₂ or OR7; where n is an integer number from 1-3;
R5 and R6 can also be independently selected from amino acids, natural or unnatural attached to thienopyrimidine core via a C-1-4 alkyl linker;
R7, R8 and R9 are each independently —H, —C1-6 alkyl, —C3-6 cycloalkyl, —C6-10 aryl, 3-10 membered heterocycle or —C1-6alkyl-C6-10aryl
R8 and R9 can also be taken together to form a 3 to 6 membered cycoalkyl;
R10 is C1-6 alkyl, —C3-6 cycloalkyl, —C6-10 aryl or 3-10 membered heterocycle.

In another aspect of the disclosure, there is provided a pharmaceutical composition comprising a compound as defined herein or a pharmaceutically acceptable salt or solvate thereof, and an acceptable excipient.

In another aspect of the disclosure, there is a compound comprising of a pro-drug particularly when R3 or R5 contain mono-phosphonate moieties, such as but not limited to CHR7PO(OH)₂; CHR7(SO₂NHR7)PO(OH)₂; (CH₂)n(SO₂NHR7)PO(OH)₂. Mono-phosphonate such as those described in this invention can be converted to pro-drugs such as those known to medicinal chemists for improving the oral bioavailability and systemic exposure of nucleotide (mono-phosphate) antitumor and antiviral drugs (for review on this topic and examples see Jordheim, L. P. et al. Nature Reviews/Drug Discovery 2013, 12, 447-464).

In another aspect of the disclosure, there is provided a method for inhibiting human farnesyl pyrophosphate synthase, comprising administering a therapeutically effective amount of a compound as defined herein or a pharmaceutically acceptable salt or solvate thereof, to a patient.

In another aspect of the disclosure, there is provided a method for inhibiting human farnesyl pyrophosphate synthase, comprising administering a combination of a therapeutically effective amount of a compound as defined herein together with an N-BP inhibitor or any other inhibitors of the human FPPS or GGPPS or a pharmaceutically acceptable salt or solvate thereof, to a patient.

In yet another aspect of the disclosure there is provided a method for treating or preventing osteoporosis, treating cancer, lowering of cholesterol, preventing or arresting the progression of neurodegenerative diseases, comprising administering a therapeutically effective amount of a compound as defined herein, or a pharmaceutically acceptable salt or solvate thereof to a patient.

In another aspect of the disclosure, there is provided the use of a compound as defined herein or a pharmaceutically acceptable salt or solvate thereof, in the manufacture of a medicament for inhibiting human farnesyl pyrophosphate synthase.

In another aspect of the disclosure, there is provided the use of a compound as defined herein or a pharmaceutically acceptable salt or solvate thereof, in the manufacture of a medicament for inhibiting human geranylgeranyl pyrophosphate synthase.

In another aspect of the disclosure, there is provided the use of a compound as defined herein or a pharmaceutically acceptable salt or solvate thereof, in the manufacture of a medicament for treating or preventing osteoporosis, bacterial infection, viral infection, infection with protozoa, cancer or lowering of cholesterol.

In another aspect of the disclosure, there is provided the use of a compound as defined herein or a pharmaceutically acceptable salt or solvate thereof, for treating or preventing osteoporosis, bacterial infection, viral infection, infection with protozoa, cancer or lowering of cholesterol.

In another aspect of the disclosure, there is provided a method for treating or preventing Alzheimer's disease, related disorders, and tauopathies using a compound as defined herein or a pharmaceutically acceptable salt or solvate thereof.

In another aspect of the disclosure, there is provided a pharmaceutical composition as defined herein for use in inhibiting human farnesyl pyrophosphate synthase.

In another aspect of the disclosure, there is provided a pharmaceutical composition as defined herein for use in inhibiting human geranylgeranyl pyrophosphate synthase.

In another aspect of the disclosure, there is provided methods for inhibiting hFPPS and directly or indirectly hGGPPS for the treatment or prevention of disease conditions associated with overexpression of these enzymes and intracellular accumulation of their corresponding metabolites (FPP) and/or (GGPP).

In one aspect, there is provided a process for preparing a compound of formula I as defined herein.

### DESCRIPTION OF THE EMBODIMENTS

In accordance with one embodiment, the disclosure provides a compound of formula I as defined above, or a pharmaceutically acceptable salt or solvate thereof, wherein

### X═O, NR4, or CHR4;

R2 is selected from H, C1-6alkyl, C3-6 cycloalkyl, C6-10aryl, 3-10 membered heterocycle, —CONHR7, —SO₂NHR7;
R3 is selected from CH[PO(OH)₂]₂; CH₂PO(OH)₂; CH(CO₂H)₂; CH(SO₂NHR7)PO(OH)₂; COCO₂H; CR8R9CO₂H; or CR8R9COR10;
R4 is independently H or C1-6alkyl;
R5 and R6 are independently selected from H, C1-6alkyl, optionally substituted C3-6 cycloalkyl, optionally substituted C6-10aryl, optionally substituted 3-10 membered heterocycle, CO₂H, CH₂CO₂H, CH₂PO(OH)₂, SO₂NR7R8, NR7R8, NH(CH₂), PO(OH)₂, or OR7;
R5 and R6 can also be independently selected from amino acids, natural or unnatural attached to thienopyrimidine core via a C-1-4 alkyl linker;
R7, R8 and R9 are each independently —H, —C1-6 alkyl, —C3-6 cycloalkyl, —C6-10 aryl, 3-10 membered heterocycle or —C1-6alkyl-C6-10aryl;
R8 and R9 can also be taken together to form a 3 to 6 membered cycoalkyl;
R10 is C1-6 alkyl, —C3-6 cycloalkyl, —C6-10 aryl or 3-10 membered heterocycle.

In accordance with one embodiment, the disclosure provides a compound of formula I as defined above, or a pharmaceutically acceptable salt or solvate thereof, wherein

### X═O, NR4, or CHR4;

R2 is selected from H, C1-6alkyl, C3-6 cycloalkyl, C6-10aryl, 3-10 membered heterocycle, —CONHR7, —SO₂NHR7;
R3 is selected from CH[PO(OH)₂]₂; CH₂PO(OH)₂; CH(CO₂H)₂; CH(SO₂NHR7)PO(OH)₂; COCO₂H; CR8R9CO₂H; or CR8R9COR10;
R4 is independently H or C1-6alkyl;
R5 is H, C1-6alkyl, phenyl, CO₂H, CH₂CO₂H, CH₂PO(OH)₂, SO₂NR7R8, NR7R8, NH(CH₂), PO(OH)₂, or OR7;
R6 is independently selected from optionally substituted C3-6 cycloalkyl, substituted phenyl, optionally substituted naphtyl, optionally substituted 3-10 membered heterocycle, CO₂H, CH₂CO₂H, CH₂PO(OH)₂, SO₂NR7R8, NR7R8, NH(CH₂), PO(OH)₂, or OR7;
R7, R8 and R9 are each independently —H, —C1-6 alkyl, —C3-6 cycloalkyl, —C6-10 aryl, 3-10 membered heterocycle or —C1-6alkyl-C6-10aryl;
R8 and R9 can also be taken together to form a 3 to 6 membered cycoalkyl;
R10 is C1-6 alkyl, —C3-6 cycloalkyl, —C6-10 aryl or 3-10 membered heterocycle.

In accordance with one embodiment, the disclosure provides a compound of formula I as defined above, or a pharmaceutically acceptable salt or solvate thereof, wherein

### X═NR4, or CHR4;

R2 is selected from H or C1-6alkyl;
R3 is selected from CR8(PO(OH)₂)₂, CR8R9CO₂H; and CR8R9PO(OH)₂;
R4 is independently H or C1-6alkyl;
R5 is H; C1-6alkyl, phenyl, CO₂H, CH₂CO₂H, CH₂PO(OH)₂, NR7R8, or OR7
R6 is independently selected from optionally substituted C3-6 cycloalkyl, substituted phenyl, optionally substituted naphtyl, optionally substituted 3-10 membered heterocycle;
R7, R8 and R9 are each independently —H, —C1-6 alkyl, —C3-6 cycloalkyl, —C6-10 aryl, 3-10 membered heterocycle or —C1-6alkyl-C6-10aryl;
R8 and R9 can also be taken together to form a 3 to 6 membered cycoalkyl.

In accordance with one embodiment, the disclosure provides a compound of formula I as defined above, or a pharmaceutically acceptable salt or solvate thereof, wherein

### X is NR4 or CHR4;

### R2 is H;

R3 is selected from CH[PO(OH)₂]₂; CH₂PO(OH)₂; or CR8R9CO₂H;
R4 is independently H or C1-6alkyl;

### R5 is H, CO₂H, NO₂ or NR7R8;

R6 is independently selected from substituted phenyl, optionally substituted naphtyl, optionally substituted 3-10 membered heterocycle;
R7, R8 and R9 are each independently —H, —C1-6 alkyl, —C3-6 cycloalkyl, —C6-10 aryl, 3-10 membered heterocycle or —C1-6alkyl-C6-10aryl;
R8 and R9 can also be taken together to form a 3 to 6 membered cycoalkyl.

In accordance with one embodiment, the disclosure provides a compound of formula I as defined above, or a pharmaceutically acceptable salt or solvate thereof, wherein

### X is NR4;

### R2 is H;

R3 is selected from CH[PO(OH)₂]₂; CH₂PO(OH)₂; or CR8R9CO₂H;

### R4 is H;

### R5 is H, CO₂H, NO₂ or NR7R8;

R6 is independently selected from substituted phenyl, optionally substituted naphthyl, and optionally substituted 3-10 membered heterocycle;
R7, R8 and R9 are each independently —H, —C1-6 alkyl, —C3-6 cycloalkyl, —C6-10 aryl, 3-10 membered heterocycle or —C1-6alkyl-C6-10aryl; preferably, when R3 is CR8R9CO₂H, one of R8 or R9 is H;
R8 and R9 can also be taken together to form a 3 to 6 membered cycoalkyl.

In one embodiment, in the compounds of formula (I), R2 is H.

In one aspect, in the compounds of formula (I), X is NR4. Preferably, X is NH.

In one embodiment, in the compounds of formula (I) X is NR4 and R3 is CR8R9-SO₂NR7(PO(OH)₂), CR8(PO(OH)₂)₂, CR8R9CO₂H; CR8R9PO(OH)₂; preferably, X is NH.

In one embodiment, in the compounds of formula (I) X is NR4 and R3 is CR8(PO(OH)₂)₂, CR8R9CO₂H; CR8R9PO(OH)₂; preferably, X is NH.

In one embodiment, in the compounds of formula (I) X is NR4 and R3 is CH₂CO₂H CH[PO(OH)₂]₂; or CH₂PO(OH)₂; preferably, X is NH.

In one embodiment, in the compounds of formula (I) X is NR4 and R3 is CR8(PO(OH)₂)₂, CR8R9CO₂H; CR8R9PO(OH)₂; preferably, X is NH.

In one embodiment, in the compounds of formula (I) X is NR4 and R3 is CH[PO(OH)₂]₂; or CH₂PO(OH)₂; preferably, X is NH.

In one embodiment, in the compounds of formula (I) X is NR4 and R3 is CR8R9CO₂H; preferably, X is NH.

In one embodiment, in the compounds of formula (I) X is CR4R4 and R3 is CR8R9-SO₂NR7(PO(OH)₂), CR8(PO(OH)₂)₂, CR8R9CO₂H; CR8R9PO(OH)₂; in CR4R4, R4 is independently H or C1-3 alkyl; or both R4 are H or C1-3 alkyl; or one of R4 is H and the other is C1-3 alkyl.

In one embodiment, in the compounds of formula (I) X is CR4R4 and R3 is CR8(PO(OH)₂)₂, CR8R9CO₂H; CR8R9PO(OH)₂ in CR4R4, R4 is independently H or C1-3 alkyl; or both R4 are H or C1-3 alkyl; or one of R4 is H and the other is C1-3 alkyl.

In one embodiment, in the compounds of formula (I) X is CR4R4 and R3 is CH₂CO₂H CH[PO(OH)₂]₂; or CH₂PO(OH)₂ in CR4R4, R4 is independently H or C1-3 alkyl; or both R4 are H or C1-3 alkyl; or one of R4 is H and the other is C1-3 alkyl.

In one embodiment, in the compounds of formula (I) X is CR4R4 and R3 is CR8(PO(OH)₂)₂, CR8R9CO₂H; CR8R9PO(OH)₂ in CR4R4, R4 is independently H or C1-3 alkyl; or both R4 are H or C1-3 alkyl; or one of R4 is H and the other is C1-3 alkyl.

In one embodiment, in the compounds of formula (I) X is CR4R4 and R3 is CH[PO(OH)₂]₂; or CH₂PO(OH)₂ in CR4R4, R4 is independently H or C1-3 alkyl; or both R4 are H or C1-3 alkyl; or one of R4 is H and the other is C1-3 alkyl.

In one embodiment, in the compounds of formula (I) X is CR4R4 and R3 is CR8R9CO₂H; in CR4R4, R4 is independently H or C1-3 alkyl; or both R4 are H or C1-3 alkyl; or one of R4 is H and the other is C1-3 alkyl.

In one embodiment, in the compounds of formula (I) R3 is CR8R9-SO₂NR7(PO(OH)₂), CR8(PO(OH)₂)₂, CR8R9CO₂H; CR8R9PO(OH)₂

In one embodiment, in the compounds of formula (I) R3 is CR8(PO(OH)₂)₂, CR8R9CO₂H; CR8R9PO(OH)₂

In one embodiment, in the compounds of formula (I) R3 is CH₂CO₂H CH[PO(OH)₂]₂; or CH₂PO(OH)₂.

In one embodiment, in the compounds of formula (I) R3 is CR8(PO(OH)₂)₂, CR8R9CO₂H; CR8R9PO(OH)₂

In one embodiment, in the compounds of formula (I) R3 is CH[PO(OH)₂]₂; or CH₂PO(OH)₂.

In one embodiment, in the compounds of formula (I) R3 is CR8R9CO₂H.

In one embodiment, in the compounds of formula (I) each R4 is H, C1-6alkyl, aryl or 3-10 membered heterocycle.

In one embodiment, in the compounds of formula (I) each R4 is H, C1-6alkyl or aryl.

In one embodiment, in the compounds of formula (I) each R4 is H, C1-3alkyl or C6aryl.

In one embodiment, in the compounds of formula (I) each R4 is independently H or C1-3 alkyl.

In one embodiment, in the compounds of formula (I) each R4 is independently H or C1-3 alkyl.

In one embodiment, in the compounds of formula (I) both R4 in CR4R4 are H, C1-6alkyl or aryl.

In one embodiment, in the compounds of formula (I) both R4 in CR4R4 are C1-6alkyl.

In one embodiment, in the compounds of formula (I) one R4 in CR4R4 is H and the other is C1-6 alkyl.

In one embodiment, in the compounds of formula (I) one R4 in CR4R4 is H and the other is C1-3 alkyl.

In one embodiment, in the compounds of formula (I), X—R3 is a natural or unnatural amino-acid.

In one embodiment, in the compounds of formula (I), X—R3 is —NH—CH[PO(OH)₂]₂.

In one embodiment, in the compounds of formula (I), X—R3 is —NH—CH₂PO(OH)₂.

In one embodiment, in the compounds of formula (I), X—R3 is —NH—CR8R9CO₂H.

In one embodiment, in the compounds of formula (I), X—R3 is —NH—CR8(PO(OH)₂)₂.

In one embodiment, in the compounds of formula (I), X—R3 is —NH—CR8R9PO(OH)₂.

In one embodiment, in the compounds of formula (I), X—R3 is —NH—CR8(PO(OH)₂)₂, —NH—CR8R9CO₂H; or —NH—CR8R9PO(OH)₂.

In one embodiment, in the compounds of formula (I), X—R3 is NH—CR8R9CO₂H, wherein R8 and R9 are independently H, alkyl, alkyl-aryl or together form a cycloalkyl.

In one embodiment, in the compounds of formula (I) R5 is H.

In one embodiment, in the compounds of formula (I) R5 is NR7R8.

In one embodiment, in the compounds of formula (I) R6 is optionally substituted phenyl.

In one embodiment, in the compounds of formula (I) R6 is substituted phenyl.

In one embodiment, in the compounds of formula (I) R6 is a phenyl substituted one or more time by halogen, C1-6alkyl, C1-6 cycloalkyl, C2-6alkenyl, C2-6alkynyl, C1-6 alkoxy, C2-6alkenyloxy, C2-6alkynyloxy, C1-6 cycloalkoxy, —NR40R41, —C(O)NR40R41, —NR40COR41, carboxy, azido, cyano, hydroxyl, nitro, —OR40, —SR40, —S(O)0-2R40, —C(O)R40, —C(O)OR40 and —SO2NR40R41; wherein R40 and R41 are each independently H, halogen, C1-6alkyl, C2-6alkenyl or C2-6alkynyl.

In one embodiment, in the compounds of formula (I) R6 is a phenyl substituted one or more time by halogen, C1-6alkyl, C1-6 cycloalkyl, C1-6 alkoxy, or C1-6 cycloalkoxy.

In one embodiment, in the compounds of formula (I), R6 is optionally substituted 3-10 membered heterocycle.

In one embodiment, in the compounds of formula (I), one of R5 or R6 is H and the other is a substituted aryl. Preferably, the aryl is a naphthyl or substituted phenyl.

In one embodiment, in the compounds of formula (I), R5 is H, R6 is an optionally substituted phenyl, R2 is H and X—R3 is —NH—CH[PO(OH)₂]₂, —NH—CH₂PO(OH)₂ or —NH—CR8R9CO₂H wherein R8 and R9 are independently H, methyl, ethyl, isopropyl, benzyl or together form a cyclopropyl.

In one embodiment, in the compounds of formula (I), R5 is H, R6 is an optionally substituted phenyl, R2 is H and X—R3 is —NH—CH[PO(OH)₂]₂, —NH—CH₂PO(OH)₂ or —NH—CR8R9CO₂H wherein R8 and R9 are independently H, methyl, ethyl, isopropyl, benzyl or together form a cyclopropyl.

In one embodiment, in the compounds of formula (I) R7 and R8 are each independently H or —C1-6 alkyl.

In one embodiment, in the compounds of formula (I) R8 and R9 are each independently —H, —C1-6 alkyl, —C6-10 aryl, or —C1-6alkyl-C6-10aryl;

In one embodiment, in the compounds of formula (I) R8 and R9 are each independently —H, —C1-6 alkyl, phenyl, or benzyl;

In one embodiment, in the compounds of formula (I) R8 is H and R9 is —C1-6 alkyl, phenyl, or benzyl;

In one embodiment, in the compounds of formula (I) R8 is H and R9 is phenyl, or benzyl

In one embodiment, in the compounds of formula (I), R6 is an optionally substituted phenyl, R2 is H and X—R3 is —NH—CH[PO(OH)₂]₂, —NH—CH₂PO(OH)₂ or —NH—CR8R9CO₂H wherein R8 and R9 are independently H, methyl, ethyl, isopropyl, benzyl or together form a cyclopropyl.

In one embodiment, in the compounds of formula (I), R6 is a substituted phenyl when X—R3 form an alpha-amino acid of general formula NH—CR8R9CO₂H.

In accordance with one embodiment, the disclosure provides a compound of formula I as defined above, or a pharmaceutically acceptable salt or solvate thereof, wherein

### X═O;

R2 is H, or C1-6alkyl;
R3 is C1-6alkyl;

### R5 is H;

R6 is independently selected from optionally substituted C3-6 cycloalkyl, substituted phenyl, optionally substituted naphtyl, optionally substituted 3-10 membered heterocycle, CO₂H, CH₂CO₂H, CH₂PO(OH)₂, SO₂NR7R8, NR7R8, NH(CH₂), PO(OH)₂, or OR7;
R7, R8 and R9 are each independently —H, —C1-6 alkyl, —C3-6 cycloalkyl, —C6-10 aryl, 3-10 membered heterocycle or —C1-6alkyl-C6-10aryl;
R8 and R9 can also be taken together to form a 3 to 6 membered cycoalkyl.

One possible feature of the present invention is to provide novel hFPPS and/or hGGPPS inhibitors and methods for treating hFPPS-dependent or hGGPPS-dependent disorders with advantageous biopharmaceutical properties as compared to the current drugs that target the human FPPS.

The present disclosure describes molecules that are structurally different from those known in the literature to inhibit the human FPPS or any other FPPS enzyme from a microbial, mammalian, plant or a host other than the human. Some members of this class of compounds may also inhibit the human GGPPS. It is hoped that at least some compounds of the present disclosure would exhibit superior biopharmaceutical properties to those of the current N-BP clinical drugs.

In one embodiment, there is provided a method or use for treating or preventing osteoporosis, bacterial infection, viral infection, infection with protozoa, cancer or lowering of cholesterol, comprising administering a therapeutically effective amount of a compound as defined herein, or a pharmaceutically acceptable salt or solvate thereof to a patient.

In one embodiment of the disclosure the method, use or composition is for treating or preventing osteoporosis, treating cancer, lowering of cholesterol, preventing or arresting the progression of neurodegenerative diseases.

In one embodiment of the disclosure the method, use or composition is for treating or preventing osteoporosis.

In one embodiment of the disclosure the method, use or composition is for treating.

In one embodiment of the disclosure the method, use or composition is for lowering cholesterol.

In one embodiment of the disclosure the method, use or composition is for preventing or arresting the progression of neurodegenerative diseases.

In one embodiment of the disclosure the method, use or composition is for treating or preventing bacterial infection, viral infection, infection with protozoa.

At least some the compounds described herein may advantageously provide selectivity toward hFPPS which means that they may inhibit to a lesser extent other related enzymes. In one embodiment, at least some of the compounds defined herein have a selective inhibition having regard to GGPPS (geranylgeranyl pyrophosphate synthase).

The term “alkyl” represents a linear or branched moiety. Examples of “alkyl” groups include but are not limited to methyl, ethyl, propyl, isopropyl, butyl, isobutyl, sec-butyl, tert-butyl, pentyl, isopentyl, neopentyl, tert-pentyl, hexyl, isohexyl or neohexyl. The term “alkyl” is also meant to include alkyls in which one or more hydrogen atom is replaced by a halogen, ie. an alkylhalide. Examples include but are not limited to trifluoromethyl, difluoromethyl, fluoromethyl, trichloromethyl, dichloromethyl, chloromethyl, trifluoroethyl, difluoroethyl, fluoroethyl, trichloroethyl, dichloroethyl, chloroethyl, chlorofluoromethyl, chlorodifluoromethyl, dichlorofluoroethyl.

The terms “alkenyl” and “alkynyl” represent a linear or branched hydrocarbon moiety which has one or more double bonds or triple bonds in the chain. Examples of alkenyl, and alkynyl groups include but are not limited to, allyl, vinyl, acetylenyl, ethylenyl, propenyl, isopropenyl, butenyl, isobutenyl, hexenyl, butadienyl, pentenyl, pentadienyl, hexenyl, hexadienyl, hexatrienyl, heptenyl, heptadienyl, heptatrienyl, octenyl, octadienyl, octatrienyl, octatetraenyl, propynyl, butynyl, pentynyl and hexynyl.

The terms “cycloalkyl” represent a cyclic hydrocarbon alkyl and are meant to include monocyclic hydrocarbon moieties. The term “cycloalkyl” is also meant to include cycloalkyls in which one or more hydrogen atom is replaced by a halogen and preferably fluoride, ie. an cylcoalkylhalide Example of cycloalkyl include but are not limited to cyclopropyl, monofluorocyclopropyl, diflurocycloproyl cyclobutyl, cyclopentyl and cyclohexyl.

The terms “cycloalkoxy,” represent a cycloalkyl moiety, respectively, which is covalently bonded to the adjacent atom through an oxygen atom. Examples include but are not limited to clyclopropyloxy, cyclobutyloxy, cyclopentyloxy, and cyclohexyloxy.

The terms “alkoxy,” “alkenyloxy,” and “alkynyloxy” represent an alkyl, alkenyl or alkynyl moiety, respectively, which is covalently bonded to the adjacent atom through an oxygen atom. Examples include but are not limited to methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy, trifluoromethoxy and neohexyloxy.

The term “aryl” represents a carbocyclic moiety containing at least one benzenoid-type ring (i.e., may be monocyclic or polycyclic), Examples include but are not limited to phenyl, tolyl, dimethylphenyl, aminophenyl, anilinyl, naphthyl, anthryl, phenanthryl or biphenyl.

The term “aryloxy” represents an aryl moiety, which is covalently bonded to the adjacent atom through an oxygen atom. Examples include but are not limited to phenoxy, dimethylphenoxy, aminophenoxy, anilinoxy, naphthoxy, anthroxy, phenanthroxy or biphenoxy.

The term “arylalkyl” represents an aryl group attached to the adjacent atom by an alkyl, alkenyl or alkynyl. Examples include but are not limited to benzyl, benzhydryl, trityl, phenethyl, 3-phenylpropyl, 2-phenylpropyl, 4-phenylbutyl and naphthylmethyl.

The term “arylalkyloxy” represents an arylalkyl moiety, which is covalently bonded to the adjacent atom through an oxygen atom. Examples include but are not limited to benzyloxy, benzhydroxy, trityloxy, phenethyloxy, 3-phenylpropoxy, 2-phenylpropoxy, 4-phenylbutoxy and naphthylmethoxy.

The term “heterocycle” represents a 3 to 11 membered optionally substituted saturated, unsaturated, partially saturated or aromatic cyclic moiety wherein said cyclic moiety is interrupted by at least one heteroatom selected from oxygen (O), sulfur (S) or nitrogen (N). Heterocycles may be monocyclic or polycyclic rings. Heterocycles may be 3 to 6 membered monocyclic ring or 5 to 6 membered monocyclic ring. Heterocycles may be 7 to 12 membered bicyclic ring or 9 to 10 membered bicyclic ring. Examples of heterocycles include but are not limited to azepinyl, aziridinyl, azetyl, azetidinyl, diazepinyl, dithiadiazinyl, dioxazepinyl, dioxolanyl, dithiazolyl, furanyl, isooxazolyl, isothiazolyl, imidazolyl, morpholinyl, morpholino, oxetanyl, oxadiazolyl, oxiranyl, oxazinyl oxazolyl, piperazinyl, pyrazinyl, pyridazinyl, pyrimidinyl, piperidyl, piperidino, pyridyl, pyranyl, pyrazolyl, pyrrolyl, pyrrolidinyl, thiatriazolyl, tetrazolyl, thiadiazolyl, triazolyl, thiazolyl, thienyl, tetrazinyl, thiadiazinyl, triazinyl, thiazinyl and thiopyranyl, furoisoxazolyl, imidazothiazolyl, thienoisothiazolyl, thienothiazolyl, imidazopyrazolyl, cyclopentapyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, cyclopentaoxazinyl, cyclopentafuranyl, benzoxazinyl, benzothiazinyl, quinazolinyl, naphthyridinyl, quinolinyl, isoquinolinyl, benzopyranyl, pyridopyridazinyl and pyridopyrimidinyl.

“Halogen atom” is specifically a fluorine atom, chlorine atom, bromine atom or iodine atom.

The term “optionally substituted” represents at each occurrence and independently, one or more halogen, amino, amidino, amido, azido, cyano, guanido, hydroxyl, nitro, nitroso, urea, OS(O)₂Rm (wherein Rm is selected from C1-6alkyl, C6-10aryl or 3-10 membered heterocycle), OS(O)₂ORn (wherein Rn is selected from H, C1-6alkyl, C6-10aryl or 3-10 membered heterocycle), S(O)₂ORp (wherein Rp is selected from H, C1-6alkyl, C6-10aryl and 3-10 membered heterocycle), S(O)₀₋₂Rq (wherein Rq is selected from H, C1-6alkyl, C6-10aryl or 3-10 membered heterocycle), OP(O)ORsORt, P(O)ORsORt (wherein Rs and Rt are each independently selected from H or C1-6alkyl), C1-6alkyl, C6-10aryl-C1-6alkyl, C6-10aryl, C1-6 alkoxy, C6-10aryl-C1-6 alkyloxy, C6-10aryloxy, C1-6 cycloalkoxy, 3-10 membered heterocycle, C(O)Ru (wherein Ru is selected from H, C1-6alkyl, C6-10aryl, C6-10aryl-C1-6alkyl or 3-10 membered heterocycle), C(O)ORv (wherein Rv is selected from H, C1-6alkyl, C6-10aryl, C6-10aryl-C1-6alkyl or 3-10 membered heterocycle), NRxC(O)Rw (wherein Rx is H or C1-6alkyl and Rw is selected from H, C1-6alkyl, C6-10aryl, C6-10aryl-C1-6alkyl or 3-10 membered heterocycle, or Rx and Rw are taken together with the atoms to which they are attached to form a 3 to 10 membered heterocycle) or SO2NRyRz (wherein Ry and Rz are each independently selected from H, C1-6alkyl, C6-10aryl, C3-10heterocycle or C6-10aryl-C1-6alkyl). In another embodiment, the term “optionally substituted” represents halogen, C1-6alkyl, C2-6alkenyl, C2-6alkynyl, C1-6 alkoxy, C2-6alkenyloxy, C2-6alkynyloxy, C1-6 cycloalkoxy, NR40R41, —C(O)NR40R41, —NR40COR41, carboxy, azido, cyano, hydroxyl, nitro, nitroso, —OR40, —SR40, —S(O)₀₋₂R40, —C(O)R40, —C(O)OR40 and —SO₂NR40R41; wherein R40 and R41 are each independently H, halogen, C1-6alkyl, C2-6alkenyl or C2-6alkynyl.

The term “independently” means that a substituent can be the same or a different definition for each item.

The excipient(s) must be “pharmaceutically acceptable” in the sense of being compatible with the other ingredients of the formulation and not being deleterious to the recipient thereof.

In one embodiment, compounds as defined herein also include prodrugs. The term “prodrug” as used herein refers to a derivative of said compound which may be in an inactive or less active form and that, when administered to a biological system, generates or liberates the biologically active compound as a result of spontaneous chemical reaction(s), enzyme catalyzed chemical reactions(s), metabolic chemical reaction(s) or a combination thereof.

As used herein, the term “hFPPS-dependent disorders” and/or “hGGPPS-dependent” is used in its non-limiting sense to describe any disease that is dependent on up-regulation of either the hFPPS or the hGGPPS enzymatic/catalytic activity.

As used herein, the term “biopharmaceutical properties” is used in its non-limiting sense to describe biopharmaceutical properties that may include, but are not limited to, properties such as enhanced efficacy, oral bioavailability, cell-membrane permeability and distribution into soft tissues, better tolerability and/or safety profile and/or pharmacokinetic properties.

In another embodiment, the present invention provides a combination comprising a therapeutically effective amount of a compound, as defined herein, and a therapeutically effective amount of at least one or more therapeutic agents useful in the method of the present disclosure.

It will be clear to a person of ordinary skill that if a further additional therapeutic agent is required or desired, ratios will be readily adjusted. It will be understood that the scope of combinations described herein is not particularly limited, but includes in principles any therapeutic agent useful for the prevention and treatment of osteoporosis (including but not limited to alendronate, risedronate or zoledronate), cancer (including but not limited to imatinib, taxol, cisplatin, doxorubicine, vinblastine, zoledronate and/or in conjunction with antimetastatic agents, antiangionevic agents such as avastatin, and antiapoptotic compounds such as Valcade), viral infection (for example in the treatment of HIV, the combination could include, inhibitors of virally encoded enzymes such as nucleoside or non-nucleoside reverse transcriptase inhibitors, protease inhibitors, integrase inhibitors, or inhibitors of viral fusion, entry inhibitors or any other step of the viral life cycle), bacterial infection, infection with protozoa or lowering of cholesterol. For immunomodulation, the combination may include NDAIDS, glucocorticoids or methotrexate.

It will be appreciated that the amount of a compound of the invention required for use in treatment will vary not only with the particular compound selected but also with the route of administration, the nature of the condition for which treatment is required and the age and condition of the patient and will be ultimately at the discretion of the attendant physician. Generally, the amount administered will be empirically determined, typically in the range of about 10 μg to 1000 mg/kg body weight of the recipient.

The desired dose may conveniently be presented in a single dose or as divided dose administered at appropriate intervals, for example as two, three, four or more doses per day.

Pharmaceutical compositions include, without limitation, those suitable for oral, (including buccal and sub-lingual), transdermal, or parenteral (including intramuscular, sub-cutaneous and intravenous) administration or in a form suitable for administration by inhalation.

The formulations may, where appropriate, be conveniently presented in discrete dosage units and may be prepared by any of the methods well known in the art of pharmacy. The methods for preparing a pharmaceutical composition can include the steps of bringing into association the compound as defined herein and pharmaceutically acceptable excipients and then, if necessary, shaping the product into the desired formulation, including applying a coating when desired.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds and combinations as defined herein may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilisation from solution, for constitution with a suitable vehicle, e.g. sterile water or saline, before use.

Compositions suitable for topical administration in the mouth include lozenges comprising the active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

For administration by inhalation, the compounds and combinations as defined herein may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

The compounds as defined herein may include a chiral center which gives rise to enantiomers. The compounds may thus exist in the form of two different optical isomers, that is (+) or (−) enantiomers. All such enantiomers and mixtures thereof, including racemic or other ratio mixtures of individual enantiomers, are included within the scope of the invention. The single enantiomer can be obtained by methods well known to those of ordinary skill in the art, such as chiral HPLC, enzymatic resolution and chiral auxiliary derivatization.

It will also be appreciated that the compounds in accordance with the present disclosure can contain more than one chiral centre. The compounds of the present invention may thus exist in the form of different diastereomers. All such diastereomers and mixtures thereof are included within the scope of the invention. The single diastereomer can be obtained by method well known in the art, such as HPLC, crystalisation and chromatography.

There is also provided pharmaceutically acceptable salts of the compounds of the present invention. What is meant by the term pharmaceutically acceptable salts of the compounds is that they are derived from pharmaceutically acceptable inorganic and organic acids and bases. Examples of suitable acids include but are not limited to hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not in themselves pharmaceutically acceptable, may be useful as intermediates in obtaining the compounds of the invention and their pharmaceutically acceptable acid addition salts. Salts derived from appropriate bases include alkali metal, alkaline earth metal or ammonium salts. The salt(s) must be “acceptable” in the sense of not being deleterious to the recipient thereof.

The term “Solvate” means that compound as defined herein incorporates one or more pharmaceutically acceptable solvents including water to give rise to hydrates. The solvate may contain one or more molecules of solvent per molecule of compound or may contain one or more molecules of compound per molecule of solvent. Illustrative non-limiting examples of hydrates include monohydrate, dihydrate, trihydrate and tetrahydrate or semi-hydrate. In one embodiment, the solvent may be held in the crystal in various ways and thus, the solvent molecule may occupy lattice positions in the crystal, or they may form bonds with salts of the compounds as described herein. The solvate(s) must be “acceptable” in the sense of not being deleterious to the recipient thereof. The solvation may be assessed by methods known in the art such as Loss on Drying techniques (LOD).

It will be appreciated by those skilled in the art that the compounds in accordance with the present invention can exist in several different crystalline forms due to a different arrangement of molecules in the crystal lattice. This may include solvate or hydrate (also known as pseudopolymorphs) and amorphous forms. All such crystalline forms and polymorphs are included within the scope of the invention. The polymorphs may be characterized by methods well known in the art. Examples of analytical procedures that may be used to determine whether polymorphism occurs include: melting point (including hot-stage microscopy), infrared (not in solution), X-ray powder diffraction, thermal analysis methods (e.g. differential scanning calorimetry (DSC) differential thermal analysis (DTA), thermogravimetric analysis (TGA)), Raman spectroscopy, comparative intrinsic dissolution rate, scanning electron microscopy (SEM).

When there is a sulfur atom present, the sulfur atom can be at different oxidation levels, ie. S, SO, or SO₂. All such oxidation levels are within the scope of the present invention.

When there is a nitrogen atom present, the nitrogen atom can be at different oxidation levels, ie. N or NO. All such oxidation levels are within the scope of the present invention.

In another embodiment, there is provided a compound of formula I selected from

| # | Structure |
|---|---|
| 1-1 | 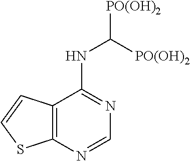 |
| 2-1 | 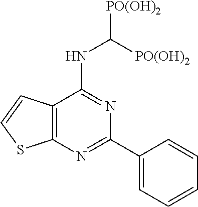 |
| 3-1 | 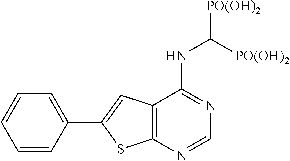 |
| 4-1 | 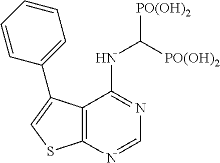 |
| 5-1 | 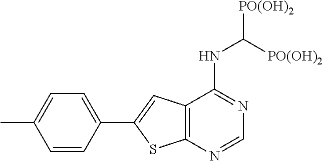 |
| 6-1 | 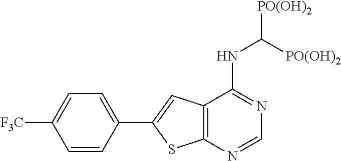 |
| 7-1 | 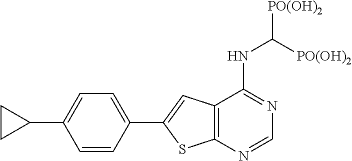 |
| 8-1 | 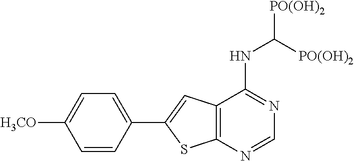 |
| 9-1 | 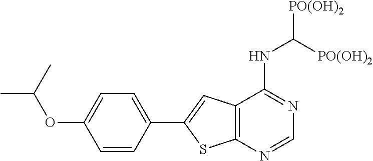 |
| 10-1 | 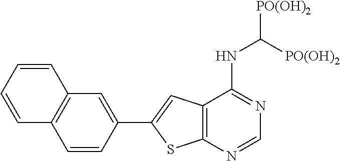 |
| 11-1 | 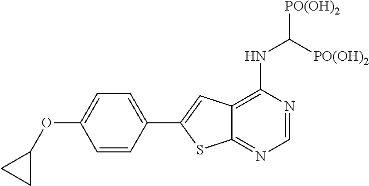 |
| 12-1 | 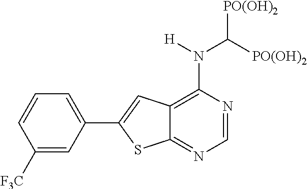 |
| 13-1 | 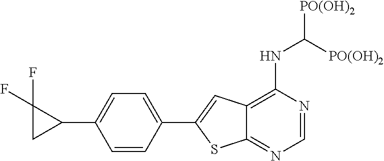 |
| 14-1 | 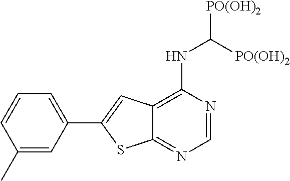 |
| 15-1 | 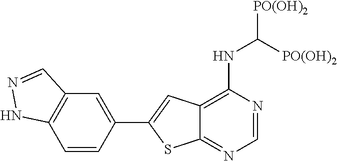 |
| 16-1 | 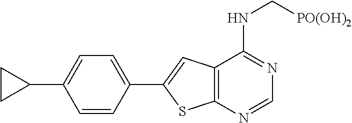 |
| 17-1 | 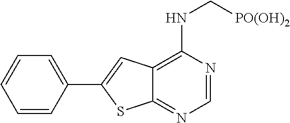 |
| 18-1 | 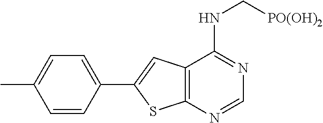 |
| 19-1 | 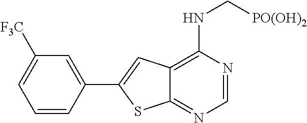 |
| 20-1 | 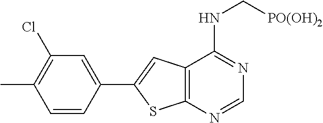 |
| 21-1 | 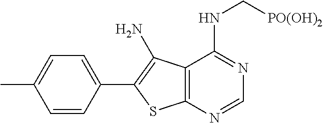 |
| 22-1 | 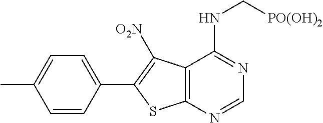 |
| 23-1 | 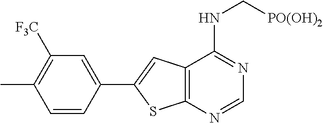 |
| 24-1 | 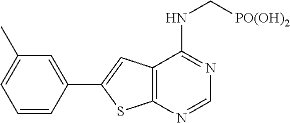 |
| 25-1 | 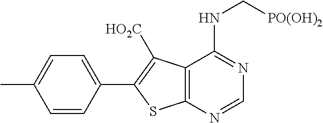 |
| 26-1 | 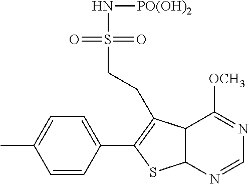 |
| 27-1 | 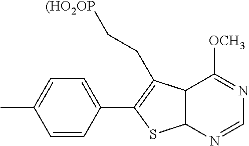 |
| 28-1 | 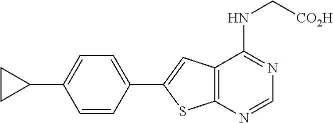 [SMILES: O=C(O)CNc1ncnc2sc(-c3ccc(C4CC4)cc3)cc12] |
| 29-1 | 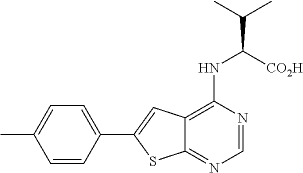 [SMILES: Cc1ccc(-c2cc3c(N[C@H](C(=O)O)C(C)C)ncnc3s2)cc1] |
| 30-1 | 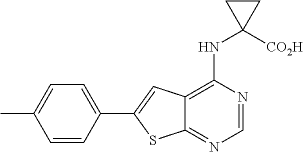 [SMILES: Cc1ccc(-c2cc3c(NC4(C(=O)O)CC4)ncnc3s2)cc1] |
| 31-1 | 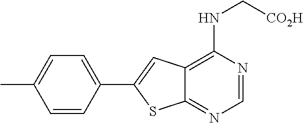 [SMILES: Cc1ccc(-c2cc3c(NCC(=O)O)ncnc3s2)cc1] |
| 32-1 | 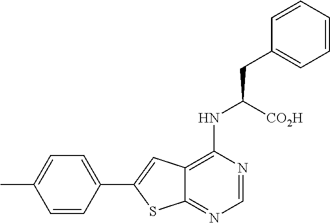 [SMILES: Cc1ccc(-c2cc3c(N[C@@H](Cc4ccccc4)C(=O)O)ncnc3s2)cc1] |
| 33-1 | 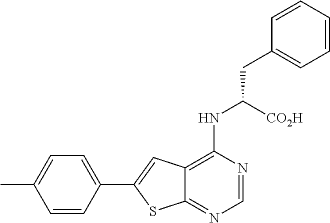 [SMILES: Cc1ccc(-c2cc3c(N[C@H](Cc4ccccc4)C(=O)O)ncnc3s2)cc1] |
| 34-1 | 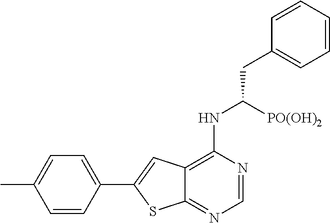 |
| 35-1 | 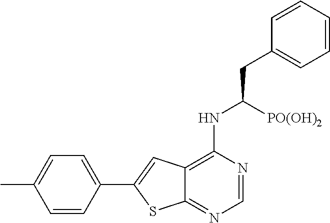 |
| 36-1 | 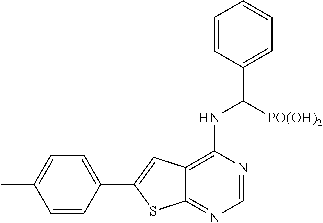 |
| 37-1 | 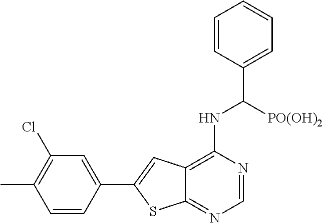 |
| 38-1 | 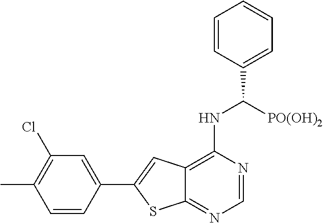 |
| 39-1 | 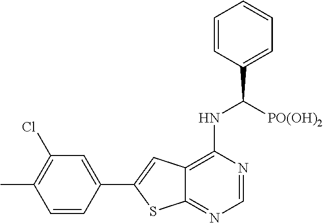 |
| 40-1 | 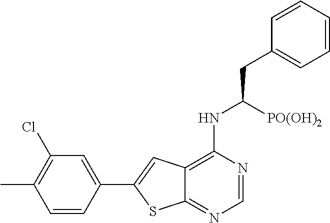 |
| 41-1 | 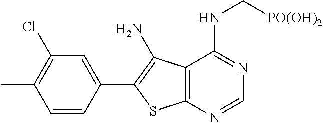 |
| 42-1 | 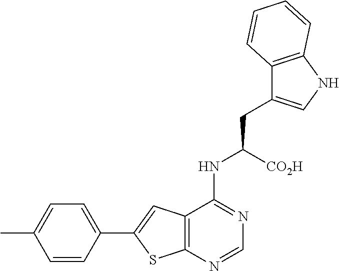 [SMILES: Cc1ccc(-c2cc3c(N[C@@H](Cc4c[nH]c5ccccc45)C(=O)O)ncnc3s2)cc1] |
| 43-1 | 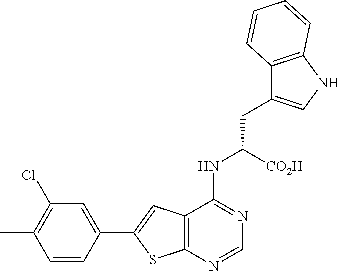 [SMILES: Cc1ccc(-c2cc3c(N[C@H](Cc4c[nH]c5ccccc45)C(=O)O)ncnc3s2)cc1Cl] |
| 44-1 | 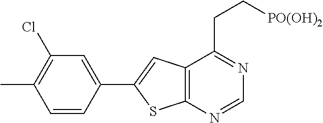 |
| 45-1 | 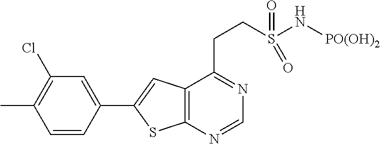 |

or a pharmaceutically acceptable salt or solvate thereof.

### ABBREVIATIONS USED IN THE DESCRIPTION OF THE PREPARATION OF THE COMPOUNDS OF THE PRESENT DISCLOSURE

| | |
|---|---|
| Bu | Butyl |
| CDCl₃ | Deuterated chloroform |
| DCM | Dichloromethane |
| DME | Dimethylether |
| DMEM | Dulbecco's Modified Eagle Medium |
| DMF | N,N-Dimethylformamide |
| DMSO | Dimethyl sulfoxide |
| Et | Ethyl |
| EtOAc | Ethyl acetate |
| HMQC | Heteronuclear multiple quantum |
| | coherence |
| HRMS | High resolution mass spectrum |
| IBX | 2-iodoxybenzoic acid |
| Me | Methyl |
| MeOH | Methanol |
| NEt₃ | Triethylamine |
| NFSI | N-fluorobenzenesulfonimide |
| NMR | Nuclear magnetic resonance |
| NMO | 4-methylmorpholine-N-oxide |
| Ph | Phenyl |
| RT | Room temperature |
| RBF | Round bottom flask |
| THF | Tetrahydofuran |
| TEA | Triethyl amine |
| TBAF | Tetra-n-butylammonium |
| | fluoride |
| TMSBr | Bromotrimethylsilane |

### Preparation of the Compounds of the Invention

The compounds of the present disclosure can be prepared according to the procedures denoted in the following reaction Schemes and Examples or modifications thereof using readily available starting materials, reagents, and conventional procedures or variations thereof well-known to a practitioner of ordinary skill in the art of synthetic organic chemistry. Specific definitions of variables in the Schemes are given for illustrative purposes only and are not intended to limit the procedures described.

The thieno[2,3-d]pyrimidin-4-amine core can be made in several ways, including via intermediate 3 as illustrated in Scheme 2. Cross-coupling of either the bromo intermediate 4 or the iodo intermediates 7b and 6 using suitable coupling fragments and catalysts, including but not limited to cross coupling reactions using Suzuki, Stille, Neghishi, Buchwald-Hartwig, Sonogashira and many other metal-catalyzed conditions (for a recent review article summarizing these types of reaction refer to Corbet, J.-P. and Mignani, G. Chem. Rev. 2006, 106, 2651-2710) leads to substitution at C-5 and/or C6 of the thieno[2,3-d]pyrimidin-4-amine core of fragments with general structure 8. The reaction conditions are summarized under Scheme 2 and more details are given as part of the preparation of key fragments and specific examples.

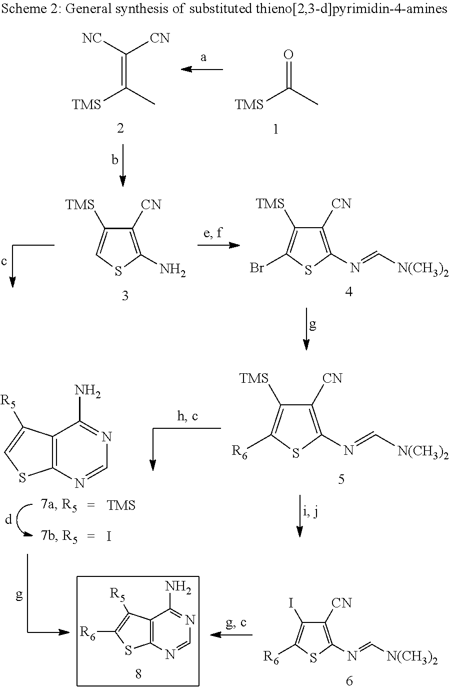

Conditions: (a) CH₂(CN)₂, NH₄OAc, AcOH, C₆H₆, Dean-Stark trap, 95° C., 24 h (90%); (b) S₈, Et₂NH, pyridine, RT, 18 h (85%); (c) HCONH₂, 130° C., 48 h (75-85%); (d) IC1, DCM, −10° C. (>98%); (e) (CH₃O)₂CHN(CH₃)₂, DMF, RT, 4 h (90%); (f) NBS, DMF, RT, 13 h in the dark (80%); (g) various cross-coupling reactions under standard Suzuki, Buchwald-Hartwig, Sonogashira and Stille conditions (isolated yields varied from 50% to 95%, none of these reactions were individually optimized); (h) TBAF, THF, 0° C. to RT, 3 h (>95%); (i) CF₃CO₂Ag, THF, −78° C., 15 min; (j) I₂, THF, −78° C., 3 h (>98%).

As illustrated in Scheme 3, thieno[2,3-d]pyrimidin-4-amines of general structure 8, generated above from Scheme 2, can be coupled to a variety of other synthetic building blocks prepared according to the procedures denoted in the following reaction Schemes 3 and Examples or modifications thereof using readily available starting materials, reagents, and conventional procedures or variations thereof well-known to a practitioner of ordinary skill in the art of synthetic organic chemistry. For example, coupling with diethyl(iodomethyl)phosphonate, followed by hydrolysis of the ethyl groups with TMSBr and MeOH, can give the mono-phosphonate derivative 9. An alternative protocol for making mono-phosphonates such as 9 is shown in Scheme 4 [i.e. general structure 17, where R═H and X═PO(OH)₂]; in our hands, this protocol provides better yields. The synthesis of bisphosphonates with general structure 10 was achieved following the protocol we reported previously (see Lin, Y.-S. et al. J. Med. Chem. 2012, 55, 3201-3215), as shown in Scheme 3.

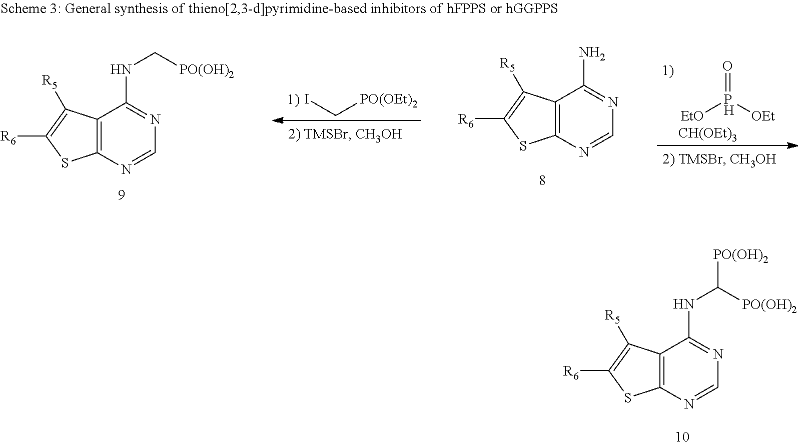

Synthesis of highly substituted thieno[2,3-d]pyrimidine inhibitors can also be achieved starting from 2,5-dihydroxy-1,4-dithiane, following literature procedures; examples include Trangerg, C. E. et al J. Med. Chem. 2002, 45, 382-389; and Hesse, S. et al. Tetrahedron Lett. 2007, 48, 5261-5264. Scheme 4 outlines the synthesis of the 6-bromo-4-chlorothieno[2,3-d]pyrimidine (14) intermediate. Subsequently, displacement of the 4-chloro moiety by a nucleophilic group (including but not limited to the amino moiety of an amino acids or an aminophosphonic acid, with the phosphonate group appropriately protected, for example as the diethyl ester) provide intermediate 15 which is amenable to a variety of cross-coupling reactions at C-6 using Suzuki, Stille, Neghishi, Buchwald-Hartwig, Sonogashira and many other metal-catalyzed conditions; for examples refer to Ghorab et al., Heteroatom Chem., 2004, 15, 57-56). Further structural modifications are possible following a number of reaction pathwayssuch as the examples shown in Scheme 4. Alternatively, the cross-coupling reaction can be performed first using intermediate 13 followed by chlorination at C-4 to give intermediate 16, which can then be reacted via an S_{N}Ar mechanism with different nucleophilic substituted amines to give 17 (Scheme 4)

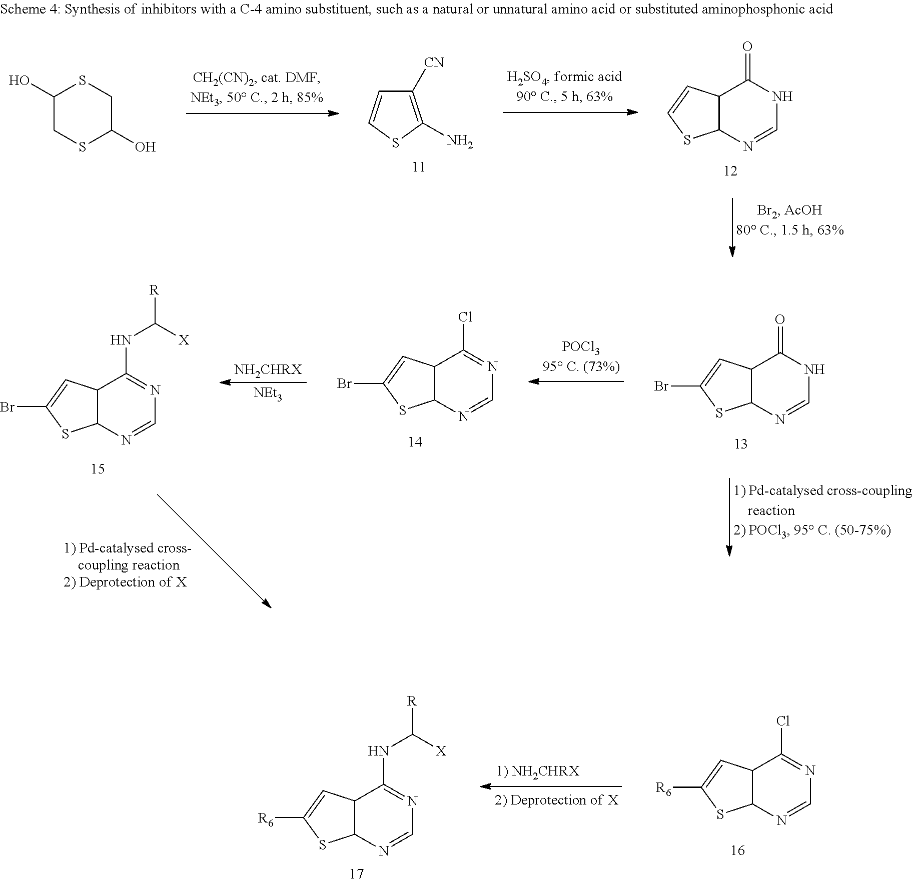

Synthesis of thieno[2,3-d]pyrimidine inhibitors with an amino substituent at C-5 can also be achieved via Buchwald-Hartwig amination of the iodo intermediate 7b (Scheme 2) and also from the 4a,7a-dihydrothieno[2,3-d]pyrimidin-4(3H)-one 12 (from Scheme 4) using the synthetic protocols shown in Scheme 5. For example, the C-5 NH₂ moiety of intermediate 24 provides a convenient precursor for modifications, which include but are not limited to alkylation of the amine or coupling to a carboxylic acid to give an amide bond, using protocols known to those skilled in the art of organic synthesis.

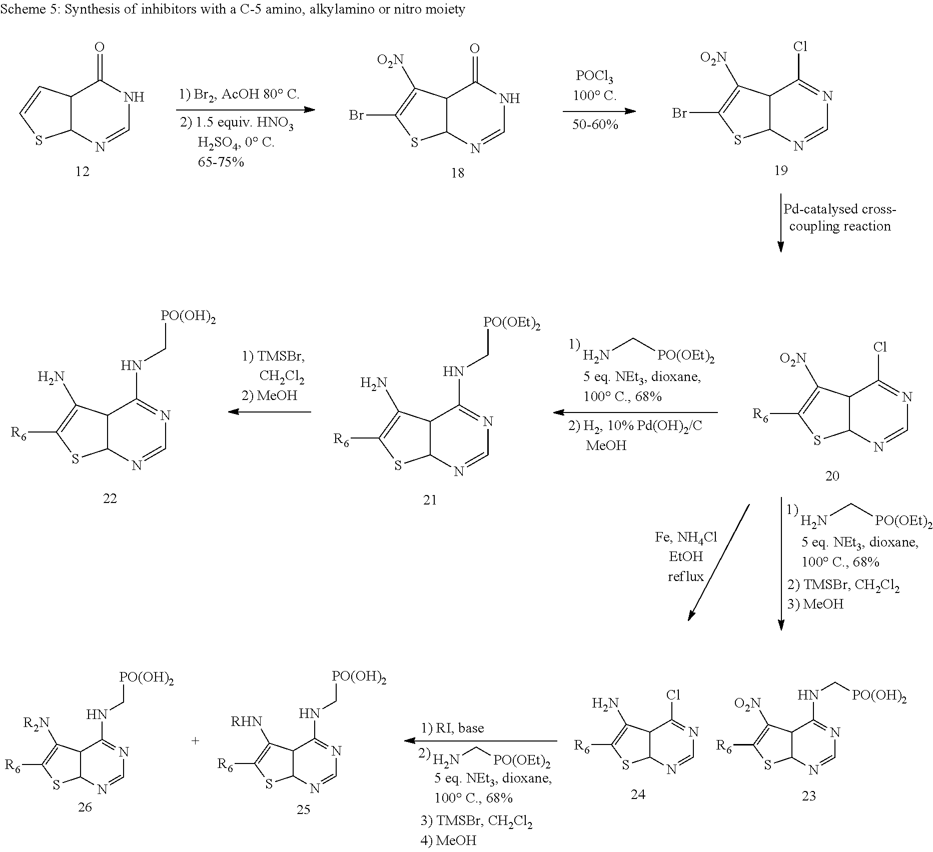

Alternatively, the synthesis of thieno[2,3-d]pyrimidine inhibitors with a carbon substituent at C-5, can also be achieved from the methyl 2-amino-4-(hydroxymethyl)thiophene-3-carboxylate 27 as shown in Scheme 6, following protocols known to those skilled in the art of organic synthesis. Pd-catalyzed cross-coupling reactions at C-6 can involve such known reactions as Suzuki, Stille, Neghishi, Buchwald-Hartwig, Sonogashira and many other metal-catalyzed conditions. Oxidation of an alcohol to the aldehyde or carboxylic acid under mild conditions can be carried out using various reagents and protocols, such as IBX for making the aldehyde 30 or the Pinnick oxidation (see Wong, L. S. and Sherburn, M. S. Org Lett. 2003, 5, 3603-3606 and references therein), trichloroisocyanutic acid/TEMPO oxidation (De Luca, L. and Giacomelli, G. J. Org. Chem. 2003, 68, 4999-5001) or tetra-n-propylammonium peruthenate in the presence of NMO-H₂O mixture (see Schmidt, A.-K. C. and Stark, C. B. W Org. Lett 2011, 13, 4164-4167) for making the carboxylic acid analog 35.

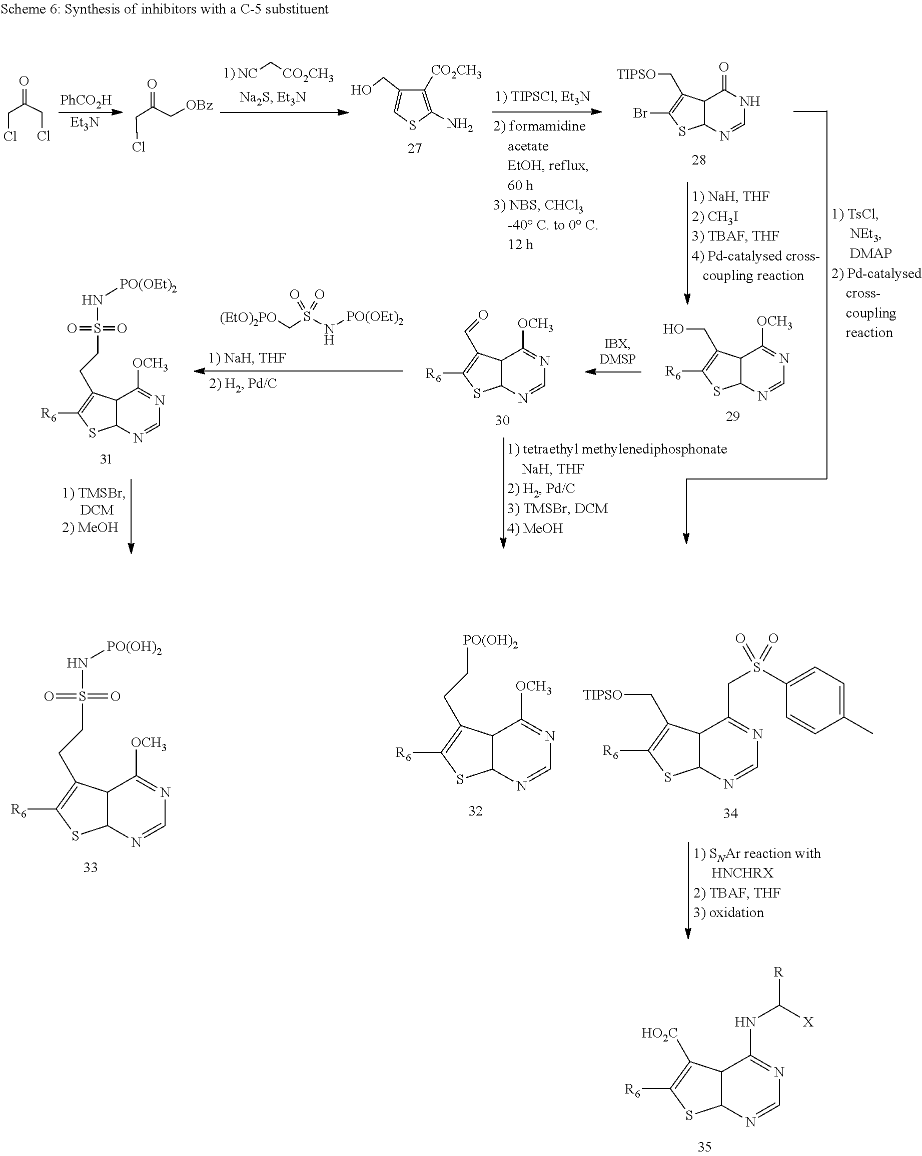

Intermediate 16 (from Scheme 4) was also used to prepare thieno[2,3-d]pyrimidine inhibitors with a carbon linker at C-4 using protocols known to those skilled in the art of organic synthesis as shown in Scheme 7.

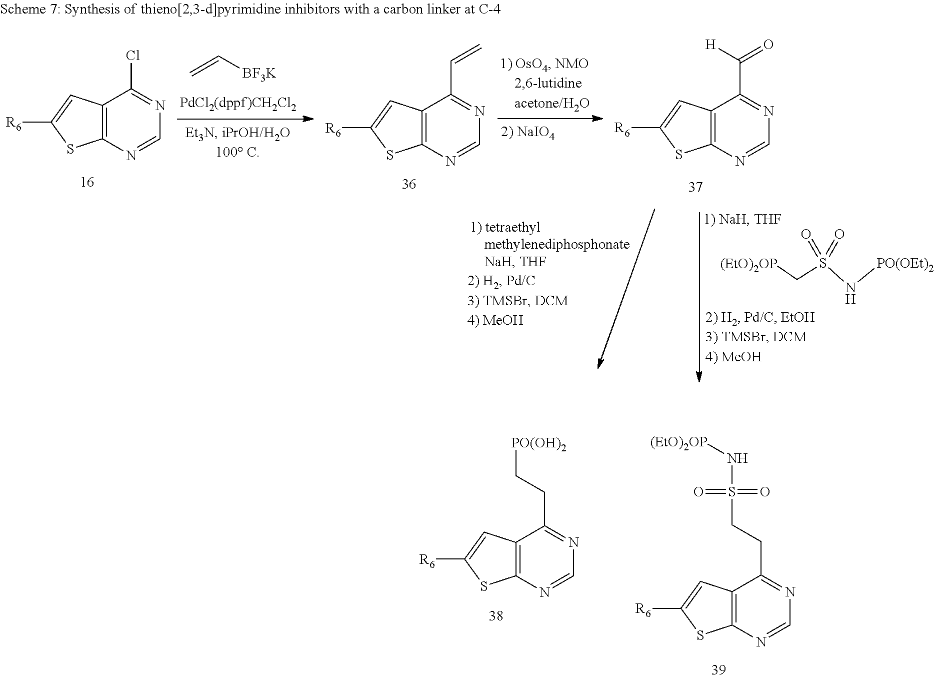

The following examples are provided to further illustrate details for the preparation and use of the compounds of the present invention. They are not intended to be limitations on the scope of the instant invention in any way, and they should not be so construed. Furthermore, the compounds described in the following examples are not to be construed as forming the only genus that is considered as the invention, and any combination of the compounds or their moieties may itself form a genus. Those skilled in the art will readily understand that known variations of the conditions and processes of the following preparative procedures can be used to prepare these compounds. All temperatures are in degrees Celsius unless noted otherwise.

### General Information:

All intermediate and final compounds were purified by normal phase flash column chromatography on silica gel using a CombiFlash instrument and the solvent gradient indicated. The purified compounds were analyzed for homogeneity by HPLC; homogeneity was confirmed by C18 reversed phase HPLC, using a Waters ALLIANCE® instrument (e2695 with 2489 UV detector and 3100 mass spectrometer), equipped with a Waters Atlantis T3 C18 5 μm column using the following conditions:

Solvent A: H₂O, 0.1% formic acid
Solvent B: CH₃CN, 0.1% formic acid
Mobile phase: linear gradient from 95% A and 5% B to 5% A and 95% B in 13 min, then 2 min at 100% B
Flow rate: 1 mL/min

All intermediates and final products were characterized by ¹H and ¹³C (1D and 2D) NMR, as well as MS; key compounds were further characterized by HR-MS. Chemical shifts (δ) are reported in ppm relative to the internal deuterated solvent, unless indicated otherwise. High-Resolution MS spectra were recorded at the McGill University, MS facilities using electrospray ionization (ESI^{+/-}).

### Synthesis of Key Fragments

### 2-(1-(Trimethylsilyl)ethyl)malononitrile (2)

Acetyltrimethylsilane (1.460 g, 12.56 mmol), malononitrile (1.140 g, 12.56 mmol) and ammonium acetate (262.1 mg, 2.386 mmol) were dissolved in acetic acid (0.58 mL, 10.05 mmol) and benzene (30 mL) in a 100 mL round bottom flask attached to a Dean-Stark trap and filled with benzene. The reaction mixture was stirred and heated to 95° C. for 24 h. The resulting orange solution was cooled and diluted with ethyl acetate (20 mL). The organic layer was washed with saturated sodium bicarbonate solution (15 mL), water (45 mL), brine (15 mL) and dried over MgSO₄. The product was purified by column chromatography (25% ethyl acetate/hexanes) to give the desired product as clear pale yellow oil in 90% yield (1.973 g).

¹H NMR (400 MHz, CDCl₃) δ 2.338 (s, 3H), 0.353 (s, 9H) ¹³C NMR (75 MHz, CDCl₃) δ=188.0, 113.0, 111.2, 94.3, 24.1, −2.33 MS (ESI⁻) m/z: 163.1 (M−H⁺)⁻.

### 2-Amino-4-(trimethylsilyl)thiophene-3-carbonitrile (3)

2-(1-(trimethylsilyl)ethyl)malononitrile (1.21 g, 7.365 mmol) and sulfur (248.02 mg, 7.734 mmol) were dissolved in pyridine (25 mL) at room temperature. To this, diethylamine (0.762 mL, 7.365 mmol) was added dropwise. The reaction mixture stirred at room temperature for 18 h. Evaporation of pyridine afforded the crude thiophene, which was dissolved in ethyl acetate (20 mL) and washed with water (45 mL), brine (15 mL), and dried over MgSO₄. Purification by column chromatography (25% ethyl acetate/hexanes, Rf=0.58) afforded the desired product as an orange oil in 85% yield (803.5 mg).

¹H NMR (400 MHz, CDCl₃) δ 6.37 (s, 1H), 4.73 (bs, 2H), 0.31 (s, 9H) ¹³C NMR (75 MHz, CDCl₃) δ 164.2, 141.0, 116.6, 116.5, 92.1, −1.45 HRMS (ESI+) calculated for C₈H₁₃N₂SSi m/z [M+H⁺]⁺: 197.05632. found m/z 197.05615.

### 5-(Trimethylsilyl)thieno[2,3-d]pyrimidin-4-amine (7a)

Fragment 7a was obtained in two different ways: (a) 2-Amino-4-(trimethylsilyl)thiophene-3-carbonitrile (400 mg, 2.04 mmol, 1 eq.) was added to formamide (8.1 mL, 200 mmol, 100 eq) in a pressure vessel. The reaction mixture was sealed and stirred at 145° C. in the dark for 16 hours. (b) 3-Cyano-4-(trimethylsilyl)thiophen-2-yl)-N,N-dimethylformimidamide (79.3 mg, 0.315 mmol) was reacted with formamide (anhydrous, 2.5 mL, 63.08 mmol) in a dry 15 mL pressure vessel. The vessel was flushed with argon and the mixture stirred at 130° C. for 45 hours. The dark red solution was diluted with ethyl acetate, washed with water (25 mL), brine (10 mL), and dried over Na₂SO₄. The crude mixture was purified by flash column chromatography (5-30% EtOAc/hexanes, dry loading) to afford the desired product as a pink solid in 80% yield

¹H NMR (400 MHz, CDCl₃) δ 8.46 (s, 1H), 7.43 (s, 1H), 5.36 (bs, 2H), 0.46 (s, 9H) ¹³C NMR (125 MHz, CDCl₃) δ 170.7, 158.7, 153.4, 133.0, 131.1, 119.7, 0.25 ²⁹Si NMR (99 MHz, CDCl₃) δ −7.399 HRMS (ESI+) calculated for C₈H₁₃N₂SSi m/z [M+H⁺]⁺: 224.06722. found m/z 224.06705.

### Synthesis of 5-iodothieno[2,3-d]pyrimidin-4-amine (7b)

A solution of 5-(trimethylsilyl)thieno[2,3-d]pyrimidin-4-amine (220.4 mg, 0.987 mmol, 1 eq.) in dichloromethane (2 mL) was stirred at −10° C. ice slurry for 5 min. 1M Iodine monochloride in dichloromethane (2.96 mL, 2.96 mmol, 3 eq) was added to the reaction mixture drop-wise and the reaction mixture was stirred at −10° C. for 30 min. Ice cooled water (30 mL) was directly added to the reaction mixture to quench the reaction. Dichloromethane (2×20 mL) at room temperature was added to the mixture and the entire mixture was filtered through a Whatman™ 5 2.5 um filter paper. The yellowish solid was washed with dichloromethane (2×10 mL) and recrystallized with methanol to give the desired product 5-iodothieno[2,3-d]pyrimidin-4-amine as a yellow colored solid. (271.9 mg, 99% yield).

¹H NMR (300 MHz, CD₃CN): δ 8.41 (1H, s), 7.88 (1H, s), 7.44 (br s, 2H) ¹³C NMR (125 MHz, Acetone-d₆) δ 159.9, 155.1, 129.0, 116.8, 106.5, 70.9 HRMS (ESI+) calculated for C₆H₄IN₃S m/z [M+H⁺]⁺: 277.92434. found m/z 277.92353.

### 3-Cyano-4-(trimethylsilyl)thiophen-2-yl)-N,N-dimethylformimidamide

To a solution of 2-amino-4-(trimethylsilyl)thiophene-3-carbonitrile (372.40 mg, 1.90 mmol) in DMF (20 mL) was added DMF-DMA (2.5 mL, 18.97 mmol). After stirring at room temperature for 4 hours, the reaction mixture was diluted with ethyl acetate, washed with water (60 mL), brine (20 mL), and dried over MgSO₄. Solvent was removed in vacuo to afford the desired product as a brown-yellow solid in 90% yield (440.1 mg; Rf=0.3, 25% EtOAc/Hex).

¹H NMR (400 MHz, CDCl₃) δ 7.72 (s, 1H), 6.63 (s, 1H), 3.10 (d, J=5.6 Hz, 6H), 0.32 (s, 9H) ¹³C NMR (125 MHz, CDCl₃) δ 168.9, 154.8, 141.6, 120.7, 117.5, 101.2, 40.7, 35.15, −1.28 ²⁹Si NMR (99 MHz, CDCl₃) δ −6.688 HRMS (ESI+) calculated for C₁₁H₁₈N₃SSi m/z [M+H⁺]⁺: 252.09852. found m/z 252.09781.

### 5-Bromo-3-cyano-4-(trimethylsilyl)thiophen-2-yl-N,N-dimethylformimidamide (4)

N-Bromosuccinimide (121.2 mg, 0.68 mmol) was added to a solution of 3-cyano-4-(trimethylsilyl)thiophen-2-yl-N,N-dimethylformimidamide (163.0 mg, 0.648 mmol) in DMF (7 mL). The yellow solution was stirred in the absence of light at room temperature for 13 hours. The mixture was diluted with ethyl acetate, washed with water (25 mL), brine (10 mL), and dried over MgSO₄. Solvent was removed in vacuo to afford the desired product as a brown-orange solid in 80% yield (177.1 mg; Rf=0.61, 25% EtOAc/Hex).

¹H NMR (400 MHz, CDCl₃) δ 7.63 (s, 1H), 3.09 (s, 6H), 0.44 (s, 9H) ¹³C NMR (125 MHz, CDCl₃) δ 168.4, 154.6, 138.9, 116.7, 106.2, 101.9, 40.8, 35.3, 0.24 HRMS (ESI+) calculated for C₁₁H₁₇N₃BrSSi m/z [M+H⁺]⁺: 330.00903. found m/z 330.00926.

### 5-Bromo-3-cyanothiophen-2-yl-N,N-dimethylformimidamide

A 1M solution of TBAF (5.13 mL) in THF was added dropwise to a solution of 5-bromo-3-cyano-4-(trimethylsilyl)thiophen-2-yl-N,N-dimethylformimidamide (4, 1.61 g, 4.89 mmol) in THF (100 mL) cooled to 0° C. The reaction mixture was warmed to room temperature and stirred in the dark for 3 h. The volume of THF was reduced in vacuo and EtOAc was added, washed with water (3×50 mL), brine (25 mL), and dried over Na₂SO₄. The desired crude product was obtained as a red oil in 95% yield (1.2 g) and used as such for the synthesis of analogs where R₅═H without any further purification.

¹H NMR (400 MHz, CDCl₃) δ 7.64 (s, 1H), 6.86 (s, 1H), 3.10 (d, J=3.6 Hz, 6H) ¹³C NMR (300 MHz, CDCl₃) δ 167.02, 154.25, 128.29, 115.15, 99.44, 96.54, 40.80, 35.17 MS (ESI+) m/z: 258.06 [M+H⁺]⁺.

### N′-(3-cyano-4-iodo-5-phenylthiophen-2-yl)-N,N-dimethylformimidamide

Silver trifluoroacetate (93.2 mg, 0.42 mmol) was added to a solution of N′-(3-cyano-5-phenyl-4-(trimethylsilyl)thiophen-2-yl)-N,N-dimethylformimidamide (69.1 mg, 0.21 mmol) in THF (20 mL) cooled to −78° C. and stirred under argon for 15 minutes. Iodine (214.2 mg, 0.84 mmol) dissolved in THF (10 mL) was added dropwise to the cold mixture and stirred in the dark at −78° C. for 4 hours. Ethyl acetate was added and the mixture was filtered through Celite™. The filtrate was washed with 2M sodium thiosulfate, brine, and dried over Na₂SO₄. The crude mixture was purified by flash column chromatography (5-30% EtOAc/hexanes, solid loading) to afford the desired product as an orange solid in 99% yield (80.0 mg).

¹H NMR (400 MHz, CDCl₃) δ 7.77 (s, 1H), 7.58-7.51 (m, 2H), 7.40 (dtd, J=6.9, 5.5, 1.5 Hz, 3H), 3.13 (s, 6H) ¹³C NMR (75 MHz, CDCl₃) δ 167.2, 154.7, 134.1, 130.7, 129.5, 128.7, 128.6, 116.8, 105.7, 78.1, 40.9, 35.3 HRMS (ESI+) calculated for C₁₄H₁₃N₃IS m/z [M+H⁺]⁺: 381.98694. found m/z 381.98667.

### General Protocol for the Suzuki Coupling Reactions

Suzuki coupling reactions were carried out using a boronic acid, boronate ester or a potassium trifluoroborate (1.5 eq.), Pd(PPh₃)₄ (0.1 eq.) and aqueous 2M Na₂CO₃ (2.5 eq) or KF (2.5 eq.) for the base. The heteroaryl halides, such as fragments 4 and 7b (Scheme 2), fragments 13 and 15 (Scheme 4) fragment 19 (Scheme 5) and fragment 28 (Scheme 6) were dissolved in toluene/ethanol (3:1) (approximate concentration of 0.1 M). The reaction mixture was degassed and flushed with Argon and stirred at 85° C. overnight or heated at 120° C. for 15-20 min in a microwave. The crude was filtered through a plug of Celite™, rinsed with 10 mL of solvent and concentrated under vacuum. The residue was purified on silica gel using a CombiFlash instrument to give the desired products (the common solvent gradient was from 2% EtOAc in hexanes to 100% EtOAc, unless otherwise indicated).

### Examples of Suzuki Products Derived from Intermediate 4 are Given Below

### N′-(3-cyano-5-phenyl-4-(trimethylsilyl)thiophen-2-yl)-N,N-dimethylformimidamide

Isolated as a beige solid in 90% yield.

¹H NMR (400 MHz, CDCl₃) δ 7.71 (s, 1H), 7.36-7.33 (m, 5H), 3.10&3.11 (2s, 6H), 0.12 (s, 9H) ¹³C NMR (75 MHz, CDCl₃) δ 167.8, 154.6, 139.1, 136.3, 135.9, 130.6, 128.5, 128.1, 118.0, 102.7, 40.7, 35.2, 0.4. HRMS (ESI+) calculated for C₁₇H₂₂N₃SSi m/z [M+H⁺]⁺: 328.12982. found m/z 328.12920.

### N′-(3-cyano-5-(4-(trifluoromethyl)phenyl)-4-(trimethylsilyl)thiophen-2-yl)-N,N-dimethylformimidamide

Isolated as a pale brown solid in 71% yield.

¹H NMR (400 MHz, CDCl₃) δ 7.72 (s, 1H), 7.61 (d, J=8 Hz, 2H), 7.46 (d, J=8 Hz, 2H), 3.12 (d, J=12 Hz, 6H), 0.14 (s, 9H) ¹³C NMR (75 MHz, CDCl₃) δ 168.4, 154.7, 139.7, 137.4, 136.8, 130.9, 125.1, 117.7, 103.0, 40.8, 35.2, 0.5 HRMS (ESI+) calculated for C₁₈H₂₁F₃N₃SSi m/z [M+H⁺]⁺: 396.11775. found m/z 396.11519.

### Examples Using Protocol (b)

### N′-(3-cyano-5-(naphthalen-2-yl)-4-(trimethylsilyl)thiophen-2-yl)-N,N-dimethylformimidamide

Isolated as an orange solid in 63% yield.

¹H NMR (400 MHz, CDCl₃): δ 7.87-7.81 (m, 5H), 7.74 (s, 1H), 7.53-7.51 (m, 2H), 7.46 (m, 1H), 3.15 (s, 3H), 3.10 (s, 3H) 0.13 (9H, s). ¹³C NMR (75 MHz, CDCl₃) δ 167.9, 154.6, 139.1, 136.6, 133.3, 133.0, 132.8, 129.6, 128.4, 128.1, 127.9, 127.7, 126.7, 126.6, 40.7, 35.2, 0.6 HRMS (ESI+) calculated for C₂₁H₂₄N₃SSi m/z [M+H]⁺: 378.14547. found m/z 378.14412.

### General Protocol for Buchwald-Hartwig Amination Reactions

The amine (5 eq.) was added to a degassed solution of an appropriate bromide, such as fragment 6 from Scheme 2 (usually on a 0.03 mmol scale), Pd₂(dba)₃ (5 mole %), XantPhos (11 mole %), and cesium carbonate (1.7 eq.) in toluene (1 mL). The vial was purged with argon and the reaction mixture stirred at 100° C. for 18 h. A second portion of Pd₂(dba)₃ (5 mole %) and XantPhos (11 mole %) were added and the reaction mixture was stirred at 100° C. for an additional 18 h. The reaction mixture was diluted with EtOAc (10 mL), washed with water (3×10 mL) and brine (10 mL), dried over Na₂SO₄, and concentrated under vacuum. The crude residue was purified by column chromatography on silica gel using a CombiFlash instrument and a solvent gradient from 2% EtOAc in hexanes to 100% EtOAc (unless otherwise indicated) to afford the desired product.

### Examples of Buchwald-Hartwig Amination Reaction Products Derived from Intermediate 6 (Scheme 2) are Given Below

### N′-(3-cyano-4-morpholino-5-phenylthiophen-2-yl)-N,N-dimethylformimidamide

Isolated beige solid in 90% yield.

¹H NMR (500 MHz, CDCl₃) δ 7.74 (s, 1H), 7.50 (d, J=7.3 Hz, 2H), 7.35 (t, J=7.5 Hz, 2H), 7.29 (d, J=7.3 Hz, 1H), 3.75-3.71 (m, 4H), 3.11 (d, J=6.0 Hz, 6H), 3.10-3.07 (m, 4H) ¹³C NMR (126 MHz, CDCl₃) δ 164.4, 153.9, 143.8, 133.6, 129.3, 128.4, 127.6, 118.4, 116.3, 96.6, 67.5, 51.7, 40.9, 35.2. MS (ESI+) m/z: 341.2 [M+H]⁺.

### General Protocol for the Cyclization of Substituted Thiophene Fragments to the Thieno[2,3-d]Pyrimidin-4-Amines Intermediate 8 (Scheme 2)

The substituted thiophene fragment (typically, 0.04 mmol) and dry formamide (excess, >200 eq.) were added to a dry 15 mL pressure vessel. The vessel was flushed with argon and the mixture stirred at 130° C. for 48 h. The dark red solution was diluted with EtOAc, washed with water (25 mL), brine (10 mL), and dried over Na₂SO₄. The crude mixture was purified by flash column chromatography (5-100% EtOAc/hexanes, solid loading) to afford the desired product.

### Examples of Cyclized Analogs of General Structure 8 (Scheme 2)

### 6-(naphthalen-2-yl)thieno[2,3-d]pyrimidin-4-amine

Isolated as a brown solid in 35% yield.

¹H NMR (500 MHz, DMSO-d₆): δ 8.28 (s, 1H), 8.18 (s, 1H), 8.15 (s, 1H), 8.06-8.01 (m, 2H), 7.96-7.94 (m, 1H), 7.84-7.83 (m, 1H), 7.62-7.53 (m, 4H) ¹³C NMR (126 MHz, DMSO-d₆) δ 165.9, 158.3, 154.3, 133.0, 132.6, 130.7, 129.0, 128.1, 127.7, 127.0, 126.7, 124.4, 123.4, 117.1, 116.3, 104.6 HRMS (ESI+) calculated for C₁₆H₁₁N₃S m/z [M+H⁺]⁺: 278.07464. found m/z 278.07371.

### 6-(4-(trifluoromethyl)phenyl)thieno[2,3-d]pyrimidin-4-amine

Isolated as a pale yellow solid in 56% yield.

¹H NMR (500 MHz, DMSO-d₆): δ 8.29 (s, 1H), 8.14 (s, 1H), 7.86 (s, 4H), 7.66 (s, 2H) ¹³C NMR (126 MHz, DMSO-d₆) δ 166.3, 158.5, 154.6, 137.1, 135.7, 128.31 (q, J=32.0 Hz), 126.3 (q, J=3.7 Hz), 124.09 (q, J=272.0 Hz), 117.8, 116.9 HRMS (ESI+) calculated for C₁₃H₈F₃N₃S [M−H]⁻: 294.03183. found m/z 294.03171.

### 6-(thiophen-3-yl)thieno[2,3-d]pyrimidin-4-amine

Isolated as a brown solid in 28% yield.

¹H NMR (400 MHz, DMSO-d₆): δ 8.24 (s, 1H), 7.78 (m, 2H), 7.73 (dd, J=5.0, 2.9 Hz, 1H), 7.50 (br s, 2H), 7.41 (dd, J=5.0, 1.4 Hz, 1H) ¹³C NMR (126 MHz, DMSO-d₆) δ 165.3, 158.1, 154.0, 134.6, 133.1, 128.3, 125.4, 121.9, 116.6, 115.3 HRMS (ESI+) calculated for C₁₀H₇N₃S₂ m/z [M+H⁺]⁺: 234.01542. found m/z 234.01433.

### 5-morpholino-6-phenylthieno[2,3-d]pyrimidin-4-amine

The crude mixture was purified by flash column chromatography (5-100% EtOAc/hexanes with 0.1% Et₃N, solid loading) to afford the desired product as a beige solid in 50% yield.

¹H NMR (500 MHz, CDCl₃) δ 8.42 (s, 1H), 7.49-7.41 (m, 5H), 3.84 (d, J=10.6 Hz, 2H), 3.60 (td, J=11.5, 2.3 Hz, 2H), 3.03 (td, J=11.6, 2.8 Hz, 2H), 2.95 (d, J=11.8 Hz, 2H), 1.60 (s, 1H) ¹³C NMR (126 MHz, CDCl3) δ 164.5, 158.8, 154.3, 138.3, 133.4, 131.2, 131.0, 129.2, 128.6, 113.9, 67.8, 53.2 HRMS (ESI+) calculated for C₁₆H₁₇ON₄S m/z [M+H⁺]⁺: 313.11176. found m/z 313.11125.

Diethyl(aminomethyl)phosphonate was synthesized according to literature procedure (see Kálmán et al. Inorg. Chem. 2007, 46, 5260)

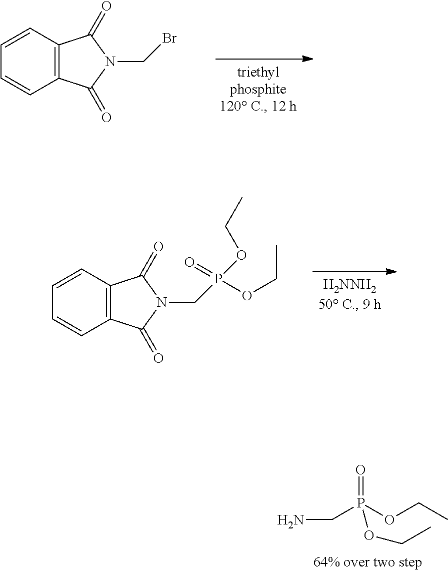

The compound was isolated as yellow liquid 64% yield (1.082 g).

¹H NMR (300 MHz, CDCl₃) δ 4.17-4.08 (m, 4H), 3.01 (d, J=10.3 Hz, 2H), 1.85 (bs, 2H), 1.34 (t, J=7.1 Hz, 6H).

### Preparation of Diethyl(Diethoxyphosphoryl)Methylsulfonylphosphoramidate

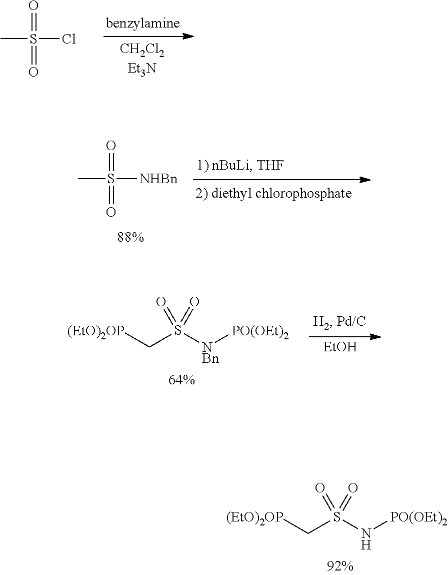

### Step 1

A solution of benzylamine (510 μL, 4.7 mmol) and Et₃N (1.37 mL, 9.8 mmol) in CH₂Cl₂ (3 mL) was cooled to 0° C., and methanesulfonyl chloride (433 μL, 5.6 mmol) was added dropwise. The mixture was stirred at room temperature for 1 hr. The mixture was diluted with EtOAc and extracted with brine. The organic layer was collected, dried over MgSO₄, concentrated, and purified by chromatography (100% Hex to EtOAc/Hex=4/1) on silica gel to give N-benzylmethanesulfonamide as white solid (763 mg, 88%). ¹H NMR (300 MHz, CDCl₃) δ 7.30-7.41 (m, 5H), 4.56 (brs, 1H), 4.33 (d, J=6.0 Hz, 2H), 2.88 (s, 3H).

### Step 2

A solution of N-benzylmethanesulfonamide (100 mg, 0.54 mmol) in THF was cooled to −78° C. and nBuLi (1.6 M in hexane, 710 μL, 1.134 mmol) was added dropwise. The mixture was warmed up to 0° C. and stirred for 1 hr. To this mixture, diethyl chlorophosphate (172 μL, 1.188 mmol) was added dropwise at 0° C. The resulting mixture was stirred at 0° C. for 1.5 hrs. The reaction was quenched by adding water. The mixture was diluted with EtOAc and extracted with brine. The organic layer was collected, dried over MgSO₄, concentrated, and purified by chromatography in silica gel (EtOAc/Hex=3/7 to 100% EtOAc) to give diethyl benzyl(((diethoxyphosphoryl)methyl)sulfonyl)phosphoramidate as light yellow oil (158 mg, 64%); MS was consistent with this products and it was used as such without further characterization.

### Step 3

A mixture of the above product (720 mg, 1.57 mmol) and Pd/C (80 mg) in MeOH (8 mL) was stirred under an atmosphere of H₂ gas overnight. The solution was filtered through Celite. The filtrate was dried under vacuum to give the desired product diethyl((diethoxyphosphoryl)methyl)sulfonylphosphoramidate as white solid (530 mg, 92%). ¹H NMR (500 MHz, CDCl₃) δ 4.20-4.28 (m, 8H), 4.05 (d, J=16.5 Hz, 2H), 1.34-1.38 (m, 12H); ¹³C NMR (125 Hz, CDCl₃) δ 64.74 (CH₂, d, J=6.3 Hz), 63.87 (CH₂, d, J=6.3 Hz), 51.10 (CH₂, d, J=137.5 Hz), 16.27 (CH₃, d, J=6.3 Hz), 16.01 (CH₃, d, J=6.3 Hz); ³¹P NMR (CDCl₃) δ 12.64, −5.89.

### General Procedure for the Synthesis of the 2-Aminopyridinyl Tetraethyl Bisphosphonic Acids of General Structure 10 from Intermediate 8 (Scheme 3)

### Step 1

In a pressure vessel, the thieno[2,3-d]pyrimidin-4-amines 8 (1 eq.), triethyl orthoformate (6 eq.), and diethylphosphite (1.2 eq.) were dissolved in toluene and stirred at 130° C. for 3 days. The solution was cooled to room temperature and the solvent was removed under vacuum. The residue was purified by silica gel chromatography on a CombiFlash instrument, using a solvent gradient from 1:1 EtOAc/Hexanes to 100% EtOAc and then to 20% MeOH in EtOAc to give the tetraethyl bisphosphonate esters in 70-85% isolated yield.

### Step 2: This Procedure was Used for all Examples Requiring the Conversion of a Tetraethyl or Diethyl Ester Bisphosphonate or Mono-Phosphonate, Respectively, to the Corresponding Free Acids

A solution of the tetraethyl bisphosphonate ester (1 eq.) in CH₂Cl₂ was cooled to 0° C. and trimethylsilyl bromide (15 eq.) was added. The reaction mixture was stirred at room temperature for 2 days (longer reaction periods of up to 5 days were required when the TMSBr reagent was old); the completion of conversion was monitored by ³¹P NMR. The mixture was then diluted with HPLC grade MeOH (˜10 mL) and stirred at RT for 1 h, the solvent was evaporated to dryness and this step was repeated four times. The organic solvents were evaporated under vacuum, the residue was dissolve in 0.5 mL MeOH, excess CH₂Cl₂ (˜10 mL) was added to induce precipitations of the final product. In cases where the final solid was colored or more like a gum than a solid, the sample was re-dissolved in HPLC grade MeOH, the flask was placed in a sonicating bath for a few minutes to assure particle dispersion and the desired product was crashed out of solution by the addition of deionized water. The solid product was collected by filtration, washed with DCM (2×) and Et₂O (2×) and dried under vacuum to give the final compound as a white powder in nearly quantitative yields.

### Synthesis of intermediate tetraethyl(((6-bromothieno[2,3-d]pyrimidin-4-yl)amino)methylene)bis(phosphonate) [15, where R═X═PO(OEt)₂)]; Scheme 4

A solution of 6-bromothieno[2,3-d]pyrimidin-4-amine (500 mg, 2.173 mmol, 1 eq.) in anhydrous toluene (20 mL) was flushed with argon in a pressure vessel. Diethylphosphite (1.96 mL, 15.2 mmol, 7 eq.) and triethyl orthoformate (0.61 mL, 3.69 mmol, 1.7 eq.) were added to the reaction mixture via syringe and the reaction mixture was argon flushed, sealed and stirred at 130° C. in the dark for 48 hours. The crude mixture was concentrated to dryness under vacuum. The crude product was purified by normal phase flash column chromatography on silica gel using a CombiFlash instrument and a solvent gradient from 20% EtOAc/hexanes to 100% EtOAc and 100% EtOAc to 20% methanol/EtOAc to give intermediate 11 as a pale yellow solid (834.2 mg, 74% isolated yield).

¹H NMR (400 MHz, CD₃OD): δ 8.44 (s, 1H), 7.85 (s, 1H), 5.96 (t, J=23.7 Hz, 1H), 4.24-4.17 (m, 8H), 1.31-1.25 (m, 12H) ¹³C NMR (75 MHz, CD₃OD): δ 168.7, 156.2, 154.5, 123.1, 119.1, 113.4, 65.2-65.0 (m), 45.6 (t, J=150.2 Hz), 16.7-16.6 (m) ³¹P NMR (81 MHz, D₂O): δ 17.2 MS (ESI−) m/z [M+H]⁺: 514.1.

### Example of Bisphosphonate Analogs of General Structure 17 (where R═X═PO(OH)₂); Scheme 4

### Example 6-1

### (((6-(4-(trifluoromethyl)phenyl)thieno[2,3-d]pyrimidin-4-yl)amino)methylene)diphosphonic acid

Isolated as a yellow solid, 25.4 mg (52% overall isolated yield).

¹H NMR (500 MHz, D₂O): δ 8.29 (s, 1H), 8.00 (s, 1H), 7.93 (d, J=8.2 Hz, 2H), 7.79 (d, J=8.2 Hz, 2H), 4.63 (t, J=18.9 Hz, 1H) ¹³C NMR (126 MHz, D₂O): δ 163.6, 156.3, 153.9, 137.5, 136.7, 129.2 (q, J=32.4 Hz), 126.2, 126.0 (q, J=3.8 Hz), 124.1 (q, J=270.1 Hz), 118.5, 116.5, Cα observed by HSQC HSQC (¹H-¹³C): ¹H δ 4.63 correlates with ¹³C δ 51.1. ³¹P NMR (81 MHz, D₂O): δ 13.6 HRMS (ESI−) calculated for C₁₄H₁₁F₃N₃P₂O₆S m/z [M−H]⁻: 467.97959. found m/z 467.9783.

### Example 7-1

### (((6-(4-cyclopropylphenyl)thieno[2,3-d]pyrimidin-4-yl)amino)methylene)diphosphonic acid

Isolated as a light brown powder 18.8 mg (47% overall isolated yield).

¹H NMR (400 MHz, D₂O): δ 8.13 (s, 1H), 7.70 (s, 1H), 7.58 (d, J=8.3 Hz, 2H), 7.11 (d, J=8.3 Hz, 2H), 4.46 (t, J=18.7 Hz, 1H), 1.88-1.84 (m, 1H), 0.93-0.86 (m, 2H), 0.66-0.62 (m, 2H) ¹³C NMR (75 MHz, D₂O): δ 165.4, 158.7, 156.0, 148.0, 141.8, 132.9, 128.7, 128.6, 121.2, 116.6, 17.1, 11.9, Cα observed by HSQC HSQC (¹H-¹³C): ¹H δ 4.46 correlates with ¹³C δ 50.9 ³¹P NMR (81 MHz, D₂O): δ 13.8 HRMS (ESI−) calculated for C₁₆H₁₆N₃F₂O₆S m/z [M−H]⁻: 440.02405. found m/z 440.02414.

### Example 8-1

### (((6-(4-methoxyphenyl)thieno[2,3-d]pyrimidin-4-yl)amino)methylene)diphosphonic acid

Isolated as a pale yellow powder; 30.8 mg (71% overall isolated yield).

¹H NMR (400 MHz, D₂O): δ 8.11 (s, 1H), 7.58 (s, 1H), 7.57 (d, J=8.0 Hz, 2H), 6.91 (d, J=8.0 Hz, 2H), 3.70 (s, 3H); central methylene proton obscured by solvent signal ¹³C NMR (126 MHz, D₂O): δ 162.7, 159.2, 156.0, 153.2, 139.1, 127.5, 126.3, 118.7, 114.6, 113.4, 55.3, Cα observed by HSQC HSQC (¹H ¹³C): ¹H δ 4.74 correlates with ¹³C δ 49.0. ³¹P NMR (81 MHz, D₂O): δ 13.7 HRMS (ESI−) calculated for C₁₄H₁₄N₃P₂O₇S m/z [M−H]⁻: 430.00332. found m/z 430.00442.

### Example 11-1

### (((6-(4-cyclopropoxyphenyl)thieno[2,3-d]pyrimidin-4-yl)amino)methylene)diphosphonic acid

Isolated as a beige powder; 16.2 mg (38% overall isolated yield).

¹H NMR (500 MHz, D₂O): δ 8.12 (s, 1H), 7.63 (s, 1H), 7.62 (d, J=8.7 Hz, 2H), 7.10 (d, J=8.7 Hz, 2H), 4.42 (br s, 1H), 3.82-3.80 (m, 1H), 0.74-0.71 (m, 2H), 0.65-0.62 (m, 2H); central methylene proton obscured by solvent signal. ¹³C NMR (126 MHz, D₂O): δ 162.7, 158.5, 156.0, 153.3, 139.1, 127.4, 126.7, 118.7, 115.7, 113.6, 51.3, 5.45, Cα observed by HSQC at ˜50.

³¹P NMR (81 MHz, D₂O): δ13.6 HRMS (ESI−) calculated for C₁₆H₁₆N₃P₂O₇S m/z [M−H]⁻: 456.0190. found m/z 456.0189.

### Example 12-1

### (((6-(3-(trifluoromethyl)phenyl)thieno[2,3-d]pyrimidin-4-yl)amino)methylene)diphosphonic acid

Isolated as a white solid, 23.4 mg (44% overall isolated yield).

¹H NMR (500 MHz, D₂O): δ 8.29 (s, 1H), 8.08 (s, 1H), 8.00 (d, J=7.8 Hz, 1H), 7.95 (s, 1H), 7.71 (d, J=7.8 Hz, 1H), 7.65 (t, J=7.8 Hz, 1H), 4.64 (t, J=18.8 Hz, 1H) ¹³C NMR (126 MHz, D₂O): δ 163.5, 156.3, 153.8, 137.7, 134.0, 130.8 (q, J=32.9 Hz), 129.7, 129.5, 124.9 (q, J=3.7 Hz), 123.9 (q, J=272.3 Hz), 122.7 (q, J=3.8 Hz), 118.5, 115.9, 50.9 ³¹P NMR (81 MHz, D₂O): δ 13.7.

HRMS (ESI−) calculated for C₁₄H₁₁F₃N₃P₂O₆S m/z [M−H]⁻: 467.98014. found m/z 467.978033.

### Example 13-1

### (((6-(4-(2,2-difluorocyclopropyl)phenyl)thieno[2,3-d]pyrimidin-4-yl)amino)methylene)diphosphonic acid

Isolated as a pale yellow solid 26.7 mg (50% overall isolated yield).

¹H NMR (500 MHz, D₂O): δ 8.27 (s, 1H), 7.86 (s, 1H), 7.75 (d, J=8.4 Hz, 2H), 7.38 (d, J=8.4 Hz, 2H), 4.63 (t, J=18.9 Hz, 1H), 2.98-2.91 (m, 1H), 1.96-1.90 (m, 1H), 1.86-1.78 (m, 1H) ¹³C NMR (126 MHz, D₂O): δ 163.2, 156.2, 153.4, 138.9, 134.3, 132.0, 128.7, 126.0, 118.5, 115.6, 113.3 (t, J=285 Hz), 26.38 (t, J=11.2 Hz), 16.1 (t, J=10.3 Hz), Cα observed by HSQC HSQC (¹H-¹³C): ¹H δ 4.63 correlates with ¹³C δ 50.5 ³¹P NMR (81 MHz, D₂O): δ 13.7.

HRMS (ESI−) calculated for C₁₆H₁₄F₂N₃P₂O₆S m/z [M−H]⁻: 476.0052. found m/z 476.0046.

### Example 15-1

### (((6-(1H-indazol-5-yl)thieno[2,3-d]pyrimidin-4-yl)amino)methylene)diphosphonic acid

Isolated as a pale pink solid, 14.9 mg (17% overall isolated yield)

¹H NMR (500 MHz, D₂O): δ 8.02 (s, 1H), 7.65 (d, J=8.2 Hz, 1H), 7.59 (s, 1H), 7.55 (s, 1H), 7.50 (d, J=8.2 Hz, 1H), 6.94 (s, 1H); central methylene proton obscured by solvent signal. ¹³C NMR (126 MHz, D₂O): δ 161.7, 155.4, 152.8, 139.2, 139.1, 133.3, 125.8, 124.3, 122.1, 118.1, 117.9, 112.9, 111.8, Cα observed by HSQC. HSQC (¹H-¹³C): ¹H δ 4.56 correlates with ¹³C δ 50.8.

³¹P NMR (81 MHz, D₂O): δ 13.9 HRMS (ESI−) calculated for C₁₄H₁₂N₅P₂O₆S m/z [M−H]⁻: 439.99890. found m/z 439.99912.

### Example of Mono-Phosphonate Analogs of General Structure 17 (where Only X═PO(OH)₂ and R═H, Alkyl, Aryl, Heteroaryl or the Side Chain of an Amino Acid); Scheme 4

### Synthesis of diethyl 4(6-bromothieno[2,3-d]pyrimidin-4-yl)amino)methyl)phosphonate (15; Scheme 4-h

The diethyl(aminomethyl)phosphonate reagent was prepared as previously reported (Kálmán, F. K. et al. Inorg. Chem. 2007, 46, 5260-5270). To a pressure vessel, 6-bromo-4-chlorothieno[2,3-d]pyrimidine (16, 1.16 g, 4.65 mmol, 1 eq.) and diethyl(aminomethyl)phosphonate (1.17 g, 6.97 mmol, 1.5 eq.) was dissolved in dioxane. Triethylamine (3.24 mL, 23.3 mmol, 5 eq.) was added drop-wise to the reaction and the pressure vessel was sealed and stirred at 100° C. for 18 hours. The reaction mixture was cooled to room temperature and diluted with ethyl acetate (50 mL). The organic layer was washed with an aqueous, saturated solution of sodium bicarbonate (15 mL), water (45 mL), brine (15 mL) and dried over anhydrous MgSO₄. The product was purified by column chromatography, (using a solvent gradient from 0%-100% ethyl acetate in hexanes and then from 0%-20% methanol in ethyl acetate) to give the desired product 15 as a yellow solid (884 mg, 50% yield).

¹H NMR (400 MHz, CD₃OD): δ 8.36 (s, 1H), 7.56 (s, 1H), 4.20-4.12 (m, 6H), 1.28 (t, J=7.1 Hz, 6H).

¹³C NMR (75 MHz, CD₃OD): δ 154.8, 122.8, 118.8, 112.7, 64.1 (d, J=6.7 Hz), 36.7 (d, J=158 Hz), 16.7 (d, J=5.9 Hz) ³¹P NMR (81 MHz, CD₃OD): δ 23.91 MS (ESI+) calculated for C₁₁H₁₅BrN₃O₃PS m/z [M+H]⁺: 380.2. found m/z 380.10.

General Protocol for S_{N}Ar reactions at the C-4 carbon of a thienopyrimidine core, such as the conversion of intermediate 14 to 15, or 16 to 17 in Scheme 4, and the conversion of intermediate 34 to 35 in Scheme 6. The following example provides a general protocol for this type of reaction:

### Synthesis of diethyl(((6-bromothieno[2,3-d]pyrimidin-4-yl)amino)methyl)phosphonate (e.g. intermediate 15, where R═H in Scheme 4)

### Step 1

To a pressure vessel, 6-bromo-4-chlorothieno[2,3-d]pyrimidine (14) (1.160 g, 4.649 mmol, 1 eq.) and diethyl(aminomethyl)phosphonate (1.165 g, 6.973 mmol, 1.5 eq.) was dissolved in dioxane. Triethylamine (3.240 mL, 23.25 mmol, 5 eq.) was added drop-wise to the reaction and the pressure vessel was sealed and stirred at 100° C. for 18 hours. The reaction mixture was cooled to room temperature and diluted with ethyl acetate (50 mL). The organic layer was washed with saturated sodium bicarbonate solution (15 mL), water (45 mL), brine (15 mL) and dried over MgSO₄. The product was purified by column chromatography on silica gel (0% to 100% ethyl acetate/hexanes and 0% to 20% methanol/ethyl acetate) to give the desired product as a yellow solid in 50% yield (883.5 mg).

¹H NMR (400 MHz, CD₃OD) δ 8.36 (s, 1H), 7.56 (s, 1H), 4.20-4.12 (m, 6H), 1.28 (t, J=7.1 Hz, 6H) ¹³C NMR (75 MHz, CD₃OD) δ 154.8, 122.8, 118.8, 112.7, 64.1 (d, J=6.7 Hz), 36.7 (d, J=158 Hz), 16.7 (d, J=5.9 Hz) ³¹P NMR (81 MHz, CD₃OD): δ 23.91 MS (ESI+) calculated for C₁₁H₁₅BrN₃O₃PS m/z [M+H]′: 380.2. found m/z 380.10.

### Step 2

Hydrolysis of the di-ethyl ester to the phosphonic acid was achieved using TMSBr, followed by MeOH as previously described.

### Example 17-1

### (((6-phenylthieno[2,3-d]pyrimidin-4-yl)amino)methyl)phosphonic acid

Isolated as a gray powder; 18.9 mg (64% overall isolated yield).

¹H NMR (500 MHz, D₂O) δ 8.00 (s, 1H), 7.55 (s, 1H), 7.48 (d, J=7.1 Hz, 2H), 7.27 (t, J=7.5 Hz, 2H), 7.20 (t, J=7.5 Hz, 1H), 3.31 (d, J=13.3, 2H) ¹³C NMR (126 MHz, D₂O) δ 162.9, 156.6 (d, J=9 Hz), 152.8, 139.8, 132.5, 129.0, 128.5, 125.5, 118.2, 114.0, 40.3 (d, J=136 Hz) ³¹P NMR (202 MHz, D₂O): δ 12.87 HRMS (ESI−) calculated for C₁₃H₁₁N₃O₃PS m/z [M−H]⁻: 320.0264. found m/z 320.0259.

### Example 18-1

### (((6-(p-tolyl)thieno[2,3-d]pyrimidin-4-yl)amino)methyl)phosphonic acid

Isolated as a white powder; 7.4 mg (24% overall isolated yield).

¹H NMR (500 MHz, D₂O) δ 7.92 (s, 1H), 7.35 (s, 1H), 7.21 (d, J=8.0 Hz, 2H), 6.90 (d, J=8.0 Hz, 2H), 3.29 (d, J=13.3, 2H), 2.10 (s, 3H) ¹³C NMR (126 MHz, D₂O) δ 162.5, 156.4 (d, J=9 Hz), 152.4, 140.0, 138.8, 129.6, 129.3, 125.2, 118.3, 113.2, 40.4 (d, J=135 Hz), 20.4 ³¹P NMR (202 MHz, D₂O): δ 12.96 HRMS (ESI−) calculated for C₁₄H₁₃N₃O₃PS m/z [M−H]⁻: 334.0415. found m/z 334.0420.

### Example 16-1

### (((6-(4-cyclopropylphenyl)thieno[2,3-d]pyrimidin-4-yl)amino)methyl)phosphonic acid

Isolated as white powder; 13.5 mg (41% overall isolated yield).

¹H NMR (500 MHz, D₂O) δ 8.09 (s, 1H), 7.47 (s, 1H), 7.33 (d, J=8.2 Hz, 2H), 6.89 (d, J=8.3 Hz, 2H), 3.47 (d, J=13.3 Hz, 2H), 1.85-1.78 (m, 1H), 1.03-0.97 (m, 2H), 0.68-0.62 (m, 2H) ¹³C NMR (126 MHz, D₂O) δ 162.3, 156.3 (d, J=9 Hz), 152.3, 144.8, 139.9, 129.4, 125.3, 125.1, 118.2, 113.1, 40.4 (d, J=136 Hz), 14.6, 9.5 ³¹P NMR (81 MHz, D₂O) δ 13.67 (s) HRMS (ESI−) calculated for C₁₆H₁₅N₃O₃PS m/z [M−H]⁻: 360.0577. found m/z 360.0562.

### Example 24-1

### (((6-(m-tolyl)thieno[2,3-d]pyrimidin-4-yl)amino)methyl)phosphonic acid

Isolated as a white powder; 8.3 mg (27% overall isolated yield).

¹H NMR (500 MHz, D₂O) δ 8.00 (s, 1H), 7.49 (s, 1H), 7.28 (d, J=7.8 Hz, 1H), 7.14 (t, J=7.6 Hz, 1H), 6.98 (d, J=7.5 Hz, 1H), 3.33 (d, J=13.2, 2H), 2.18 (s, 3H) ¹³C NMR (126 MHz, D₂O) δ 162.8, 156.5, 152.7, 139.9, 139.0, 132.4, 129.1, 128.9, 125.8, 122.5, 118.2, 113.8, 40.3 (d, J=136 Hz), 20.4 ³¹P NMR (202 MHz, D₂O): δ 13.61 HRMS (ESI−) calculated for C₁₄H₁₃N₃O₃PS m/z [M−H]⁻: 334.0421. found m/z 334.0411.

### Example 19-1

### (((6-(3-(trifluoromethyl)phenyl)thieno[2,3-d]pyrimidin-4-yl)amino)methyl)phosphonic acid

Isolated as a beige powder; 7.5 mg (21% overall isolated yield).

¹H NMR (500 MHz, D₂O) δ 8.04 (s, 1H), 7.73-7.72 (m, 2H), 7.64 (s, 1H), 7.45-7.42 (m, 2H), 3.32 (d, J=13.3, 2H) ¹³C NMR (126 MHz, D₂O) δ 163.2, 156.8, 156.7, 153.2, 138.2, 133.3, 130.4 (q, J=28 Hz), 129.6, 129.0, 124.8 (q, J=4.0 Hz), 122.0 (q, J=3.7 Hz), 118.0, 115.8, 40.3 (d, J=137 Hz) ³¹P NMR (202 MHz, D₂O): δ 13.54 HRMS (ESI−) calculated for C₁₄H₁₀F₃N₃O₃PS m/z [M−H]⁻: 388.0138. found m/z 388.0142.

### Example 20-1

### (((6-(3-chloro-4-methylphenyl)thieno[2,3-d]pyrimidin-4-yl)amino)methyl)phosphonic acid

Isolated as a pale yellow powder; 8.5 mg (25% overall isolated yield).

¹H NMR (400 MHz, D₂O) δ 8.05 (s, 1H), 7.42 (s, 1H), 7.28 (s, 1H), 7.23 (d, J=8.0 Hz, 1H), 7.05 (d, J=8.0 Hz, 1H), 3.44 (d, J=13.2 Hz, 2H), 2.19 (s, 3H) ¹³C NMR (126 MHz, D₂O) δ 162.6, 156.4, 152.5, 138.2, 136.0, 134.0, 131.5, 131.0, 125.0, 123.5, 118.0, 114.2, 40.3 (d, J=134 Hz), 19.0 ³¹P NMR (202 MHz, D₂O): δ 13.67 HRMS (ESI−) calculated for C₁₄H₁₂C1N₃O₃PS m/z [M−H]⁻: 368.0031. found m/z 368.0030.

### Example 23-1

### (((6-(4-methyl-3-(trifluoromethyl)phenyl)thieno[2,3-d]pyrimidin-4-yl)amino)methyl)phosphonic acid

Isolated as a white powder; 8.2 mg (22% overall isolated yield).

¹H NMR (400 MHz, D₂O) δ 7.95 (s, 1H), 7.46-7.43 (m, 3H), 3.34 (d, J=13.3 Hz, 2H), 2.25 (s, 3H) ¹³C NMR (126 MHz, D₂O) δ 162.6, 156.3, 156.2, 138.2, 136.3, 132.3, 130.1, 128.3, 128.0 (q, J=29.8 Hz), 124.1 (q, J=272 Hz), 121.8 (q, J=5.6 Hz), 117.9, 114.4, 40.4 (d, J=135 Hz), 18.3 ³¹P NMR (202 MHz, D₂O): δ 13.57 HRMS (ESI−) calculated for C₁₅H₁₂F₃N₃O₃PS m/z [M−H]⁻: 402.0295. found m/z 402.0286.

### Example of Mono-Phosphonate Analogs of General Structure 22 and 23; Scheme 5

### Example 21

### 1 (((5-amino-6-(p-tolyl)thieno[2,3-d]pyrimidin-4-yl)amino)methyl)phosphonic acid

### Step 1: synthesis of 6-bromo-5-nitrothieno[2,3-d]pyrimidin-4(3H)-one (18)

6-bromothieno[2,3-d]pyrimidin-4(3H)-one (13, 3.5 g, 15 mmol) was added to 10 mL of ice-cooled sulfuric acid and the suspension was vigorously stirred for 5 min and sonicated thoroughly to break up an clumps formed. Nitric acid (1 mL, 23 mmol) was carefully added dropwise at 0° C. (strong exotherm). The reaction was at RT for 30 min re-cooled to 0° C. The reaction mixture was carefully quenched with 100 mL ice-cold water, filtered and rinsed with water. The residue was collected and dried on high vacuum to furnish the desired product as a pale orange powder (2.8 g, 66%).

¹H NMR (500 MHz, DMSO-d₆) δ 13.08 (br s, 1H), 8.28 (s, 1H) ¹³C NMR (126 MHz, DMSO-d₆) δ 163.6, 154.4, 148.9, 142.0, 117.1, 108.8 MS (ESI): calcd 275.908 and 277.906 for C6H3BrN3O3S. found 276.02 and 277.99[M+H]⁺.

### Step 2

Conversion of intermediate 18 to 19 was achieved using POCl₃ following a similar protocol to that previously described in the transformation of intermediate 13 to 14 (Scheme 4)

### Step 3

Synthesis of 4-chloro-5-nitro-6-(p-tolyl)thieno[2,3-d]pyrimidine was achieved after typical Suzuki cross-coupling reaction of intermediate 19 with potassium trifluoro(p-tolyl)borate to give intermediate 20.

### Step 4 to 6

Synthesis of diethyl(((5-amino-6-(p-tolyl)thieno[2,3-d]pyrimidin-4-yl)amino)methyl)phosphonate was achieved by first S_{N}Ar displacement of the C-4 chloro of 20 with diethyl(aminomethyl)phosphonate, followed by hydrogenation of the nitro moiety using H₂ and Pd(OH)₂/C to give intermediate 21 (Scheme 5). Ester hydrolysis under the standard condition of TMSBr followed by methanolysis gave the final inhibitor, Example 21-1

Isolated as a pale yellow solid; 10 mg (90% yield).

¹H NMR (400 MHz, D₂O) δ 8.04 (s, 1H), 7.22 (d, J=8.1 Hz, 2H), 7.11 (d, J=8.1 Hz, 2H), 3.33 (d, J=13.2 Hz, 2H), 2.19 (s, 3H) ¹³C NMR (126 MHz, D₂O) δ 171.0, 160.8, 153.0, 138.3, 131.2, 129.7, 128.8, 128.4, 118.6, 113.2, 39.9 (d, J=136 Hz), 20.3 ³¹P NMR (202 MHz, D₂O): δ 13.6 HRMS (ESI−) calculated for C₁₄H₁₄N₄O₃PS m/z [M−H]⁻: 349.0530. found m/z 349.0544.

### Example of Mono-Phosphonate Analogs of General Structure 17 where R=Alkyl, Aryl, Heteroaryl or the Side Chain of an Amino Acid; Scheme 4

### Example 36-1

### (phenyl((6-(p-tolyl)thieno[2,3-d]pyrimidin-4-yl)amino)methyl)phosphonic acid

Racemic diethyl diethyl(amino(phenyl)methyl)phosphonate was prepared using the protocol reported by Wu et al. in Org. Biomol. Chem., 2006, 4, 1663-1666. However, the highly enriched R and S enantiomers are also commercially available.

### Step 1

To a round bottom flask, benzaldehyde (742.79 mg, 7.00 mmol, 1 eq.) was mixed with magnesium perchlorate (156.23 mg, 0.7 mmol, 0.1 eq.) for 15 min. Benzylamine (750 mg, 7.00 mmol, 1 eq.) and diethylphosphite (0.939 mL, 7.28 mmol, 1.04 eq.) were added the reaction mixture and heated at 85° C. for 24 hours. The crude product was dried under vacuum and loaded onto silica with ethyl acetate. The product was purified by column chromatography (0% to 100% ethyl acetate/hexanes and 0% to 20% methanol/ethyl acetate) to give the desired product as a slightly yellow transparent oil in 81% yield (1.8 g).

¹H NMR (300 MHz, CDCl₃) 7.43-7.28 (m, 10H), 4.15-3.80 (m, 7H), δ 3.56 (d, J=13.3 Hz, 1H), 2.12 (s, 1H), 1.29 (t, J=7.0 Hz, 3H), 1.14 (t, J=7.0 Hz, 3H).

### Step 2

To a round bottom flask, diethyl((benzylamino)(phenyl)methyl)phosphonate (1 g, 3.00 mmol, 1 eq.) was stirred in methanol (4 mL), Pearlman's catalyst (84.25 mg, 0.6 mmol, 0.2 eq.) was added to the reaction and flushed under argon. Hydrogen was bubbled through the reaction at RT for 3 days. The crude product was dried under vacuum and loaded onto silica with chloroform The product was purified by column chromatography (0% to 100% ethyl acetate/hexanes) to give the desired product as a transparent oil in 69% yield (504 mg).

¹H NMR (500 MHz, CDCl₃) δ 7.45 (d, J=7.7 Hz, 2H), 7.35 (t, J=7.7 Hz, 2H), 7.30 (dd, J=7.3, 2.0 Hz, 1H), 4.26 (d, J=17.1 Hz, 1H), 4.08-402 (m, 2H), 4.02-3.94 (m, 1H), 3.90-3.84 (m, 1H), 1.92 (br s, 2H), 1.27 (t, J=7.0 Hz, 3H), 1.18 (t, J=7.0 Hz, 3H).

### Step 3

Displacement of the C-4 chloro of intermediate 14 (Scheme 4) with diethyl diethyl(amino(phenyl)methyl)phosphonate via an S_{N}Ar reaction under the same conditions as previously described for the conversion of intermediate 14 to 15 (Scheme 4) gave the diethyl(((6-bromothieno[2,3-d]pyrimidin-4-yl)amino)(phenyl)methyl)phosphonate intermediate

To a pressure vessel, 6-bromo-4-chlorothieno[2,3-d]pyrimidine (13) (70 mg, 0.281 mmol, 1 eq.) and diethyl(amino(phenyl)methyl)phosphonate (136.5 mg, 0.561 mmol, 2 eq.) was dissolved in dioxane. Triethylamine (0.196 mL, 1.403 mmol, 5 eq.) was added dropwise to the reaction and the pressure vessel was sealed and stirred at 100° C. for 24 hours. The reaction mixture was cooled to room temperature and diluted with ethyl acetate (10 mL). The organic layer was washed with saturated sodium bicarbonate solution (5 mL), water (10 mL), brine (10 mL) and dried over MgSO₄. The product was purified by column chromatography (0% to 100% ethyl acetate/hexanes and 0% to 20% methanol/ethyl acetate) to give the desired product as a white solid in 36% yield (51 mg).

¹H NMR (300 MHz, CDCl₃) δ 8.42 (s, 1H), 8.11-8.07 (m, 1H), 7.70-7.67 (m, 3H), 7.27-7.22 (m, 2H), 6.32 (dd, J=22.4, 9.6 Hz, 1H), 4.31-4.07 (m, 3H), 3.93-3.84 (m, 1H), 1.28-1.19 (s, 6H). ¹³C NMR (75 MHz, CDCl₃) δ 167.7, 155.0 (d, J=9 Hz), 153.7, 128.49 (d, J=2 Hz), 128.44 (d, J=2 Hz), 127.99 (d, J=3 Hz), 121.7, 117.8, 111.3, 63.5 (d, J=7 Hz), 50.79 (d, J=156 Hz), 16.3 (dd, J=19, 6 Hz). MS (ESI+) calculated for C₁₇H₂₀BrN₃O₃PS m/z [M+H]⁺: 456.01. found m/z 456.14.

After Suzuki cross-coupling reaction, followed by ester hydrolysis following the general protocols described before inhibitors such as Examples 36-1 and 37-1 were obtained.

### Example 36-1

### (phenyl((6-(p-tolyl)thieno[2,3-d]pyrimidin-4-yl)amino)methyl)phosphonic acid

Isolated as a white solid with 52% overall yield (16 mg)

¹H NMR (400 MHz, D₂O) δ 7.72 (s, 1H), 7.58 (s, 1H), 7.48 (d, J=7.7 Hz, 2H), 7.32 (t, J=7.5 Hz, 1H), 7.20 (t, J=7.3 Hz, 1H), 7.04 (d, J=7.5 Hz, 2H), 6.57 (d, J=7.5 Hz, 2H), 4.98 (d, J=20 Hz, 1H), 1.92 (s, 3H) ¹³C NMR (126 MHz, D₂O) δ 161.7, 154.8, 151.0, 139.8, 139.1, 137.4, 128.3, 128.0, 127.0, 126.7, 125.4, 123.9, 117.3, 112.1, 54.4 (d, J=129 Hz), 19.0 ³¹P NMR (81 MHz, D₂O) δ 13.82. MS (ESI+) calculated for C₂₀H₁₇N₃O₃PS m/z [M−H]⁻: 410.08. found m/z 410.2.

### Example 37-1

### (((6-(3-chloro-4-methylphenyl)thieno[2,3-d]pyrimidin-4-yl)amino)(phenyl)methyl)phosphonic acid

Isolated as a white solid with 52% yield (15 mg)

¹H NMR (500 MHz, D₂O) δ 7.77 (s, 1H), 7.49 (d, J=7.7 Hz, 3H), 7.35 (t, J=7.2 Hz, 2H), 7.25, −7.22 (m, 1H), 7.05 (s, 1H), 6.79 (s, 1H), 6.27 (d, J=7.2 Hz, 1H), 5.00 (d, J=19.7 Hz, 1H), 1.75 (s, 3H). ¹³C NMR (126 MHz, D₂O) δ 162.8, 155.8, 152.2, 140.9, 138.4, 135.6, 131.2, 128.1, 127.8, 126.6, 124.8, 123.2, 118.1, 114.0, 110.0, 55.5 (d, J=134 Hz), 18.6. ³¹P NMR (81 MHz, D₂O) δ 13.84. MS (ESI+) calculated for C₂₀H₁₆C1N₃O₃PS m/z [M−H]⁻: 444.04. found.

### Synthesis of Analogs with a Carbon Linker at C-5 (Scheme 6)

The synthesis of the 2-amino-4-(hydroxymethyl)thiophene-3-carboxylate intermediate 27 was achieved as previously described (US 2004/0097492)

### Step 1

Intermediate 27 was first reacted with TIPSCl in the presence of base obtain the silyl ether and then cyclized with formamidine. The cyclized 5-(((triisopropylsilyl)oxy)methyl)-4a,7a-dihydrothieno[2,3-d]pyrimidin-4(3H)-one was reacted with NBS at low temperature (−40 to 0° C.) for 12 h to give intermediate 28.

### Step 2

A solution of 28 (300 mg, 0.715 mmol) in THF (3 mL) was cooled to 0° C. and NaH (34.3 mg, 0.858 mmol) was added in portions. The mixture was stirred at room temperature for 1 hr. The mixture was cooled again to 0° C. and CH₃I (58 μL, 0.930 mmol) was added, and stirring was continued at room temperature overnight. The reaction was quenched with water, diluted with EtOAc (150 mL), and extracted with brine (100 mL). The organic layer was collected, dried over MgSO₄, concentrated, and purified by chromatography (100% Hex to EtOAc/Hex=1/1) on silica gel to give 29 as white solid (115 mg, 37%). ¹H NMR (300 MHz, CDCl₃) δ 7.96 (s, 1H), 5.13 (s, 2H), 3.56 (s, 3H), 1.32-0.85 (m, 21H); ¹³C NMR (75 MHz, CDCl₃) δ 163.82, 156.69, 146.73, 136.51, 112.93, 104.99, 58.08, 34.23, 18.03, 12.11.

### Step 3

To a solution of the above intermediate (115 mg, 0.267 mmol) in THF (2 mL) at 0° C., TBAF (1M solution in THF, 293 μL, 0.293 mmol) was added. The mixture was stirred at room temperature for 2 hrs. The solvent was removed under vacuum. The residue was re-dissolved in EtOAc (100 mL), extracted with water (100 mL), dried over MgSO₄, concentrated, and purified by silica gel (20% EtAOc in Hex to 100% EtOAc) to give the deprotected alcohol (6-bromo-4-methoxythieno[2,3-d]pyrimidin-5-yl)methanol as white solid (65 mg, 89%). ¹H NMR (300 MHz, CDCl₃) δ 8.03 (s, 1H), 4.77 (s, 2H), 3.64 (s, 3H).

Various cross coupling reactions with the C-6 bromide are possible, as an example the details for the preparation of intermediate 30 (4-methoxy-6-(p-tolyl)thieno[2,3-d]pyrimidin-5-yl)methanol, which was used to prepare Example 26-1 are given below

### Step 4

In a vial, (6-bromo-4-methoxythieno[2,3-d]pyrimidin-5-yl)methanol (62 mg, 0.225 mmol), p-tolylboronic acid (61.7 mg, 0.451 mmol), and Pd(PPh)₄ (52.1 mg, 0.045 mmol) were mixed in DME (4 mL) and the mixture was flashed with argon. To this mixture, 2M potassium carbonate solution (282 μL, 0.563 mmol) was added and the solution was flushed again with argon again. The solution was stirred at 80° C. overnight. The solution was filtered through Celite. The filtrate was concentrated and purified on silica gel (10% Hex in EtOAc to 90% EtOAc in Hex) to give intermediate 29 (where R6 is a tolyl group) as brown solid (61.5 mg, 95%). ¹H NMR (300 MHz, CDCl₃) δ 8.03 (s, 1H), 7.33 (d, J=8.0 Hz, 2H), 7.24 (d, J=8.0 Hz, 2H), 5.04 (brs, 1H), 4.77 (s, 2H), 3.63 (s, 3H), 2.39 (s, 3H); ¹³C NMR (75 MHz, CDCl₃) δ 164.07, 159.49, 146.17, 138.89, 138.42, 132.87, 129.80, 129.53, 124.04, 104.98, 57.67, 34.30, 21.28.

### Step 5

To a solution of intermediate 29 (50 mg, 0.175 mmol) in DMSO (1 mL), IBX (54.5 mg, 0.262 mmol) was added and the mixture was stirred at room temperature for 3 hrs. The solution was diluted with EtOAc (100 mL) and extracted with brine (100 mL). The organic layer was collected, dried over MgSO₄, concentrated, and purified by chromatography (10% EtOAc in Hex to 100% EtOAc) on silica gel to give intermediate 30 (where R6 is totlyl) as yellow solid (40 mg, 81%). ¹H NMR (300 MHz, CDCl₃) δ 10.71 (s, 1H), 8.06 (s, 1H), 7.48 (d, J=8.1 Hz, 2H), 7.24 (d, J=8.1 Hz, 2H), 3.64 (s, 3H), 2.40 (s, 3H); ¹³C NMR (125 MHz, CDCl₃) δ 187.00, 162.60, 157.59, 149.95, 147.04, 140.05, 129.75, 129.64, 128.97, 128.51, 123.03, 34.16, 21.26.

Conversion of aldehyde intermediate 30 to compounds such as 32, 33 and 35 can be done is several ways; as an example the details for the preparation of Example 26-1 and 27-1 are given below

### Preparation of Example 26-1 from Intermediate 30 (where R6 is Tolyl)

### Step 1

A solution of diethyl((diethoxyphosphoryl)methyl)sulfonylphosphoramidate (50 mg, 0.136 mmol) in THF (3 mL) was cooled to 0° C. and NaH (10.9 mg, 0.272 mmol) was added in portions. The mixture was stirred at 0° C. for 10 min and RT for 5 min. The solution was cooled to 0° C. and a solution of intermediate 30 (38.7 mg, 0.136 mmol) in THF was added dropwise. The mixture was stirred at room temperature for 2 hrs. The reaction was quenched with MeOH. The solvent was removed under vacuum and the residue was purified by flash column chromatography (100% EtOAc to 20% MeOH in EtOAc). The product was re-suspended in EtOH, a catalytic amount of Pd/C was added and the mixture was stirred at room temperature under an atmosphere H₂ overnight. The solution was filtered through Celite. The filtrate was concentrated and purified by chromatography (100% EtOAc to 20% MeOH in EtOAc) on silica gel to give intermediate 31 (where R6 is tolyl) as white solid (35 mg, 51%). ¹H NMR (300 MHz, CDCl₃) δ 7.90 (s, 1H), 7.24 (d, J=7.7 Hz, 2H), 7.17 (d, J=7.7 Hz, 2H), 3.95-3.68 (m, 4H), 3.56 (s, 3H), 3.34 (m, 4H), 2.34 (s, 3H), 1.02 (t, J=6.8 Hz, 6H); ¹³C NMR (75 MHz, CDCl₃) δ 163.56, 158.36, 146.39, 138.42, 137.33, 130.40, 129.52, 129.36, 123.06, 61.96 (d, J=5.6 Hz), 55.22, 34.63, 23.73, 21.20, 15.94 (d, J=7.8 Hz); ³¹P NMR (81 MHz, CDCl₃) δ −1.17.

### Step 2

A solution of the above diethylester 31 (35 mg, 0.070 mmol) in CH₂Cl₂ (3 mL) was treated with TMSBr (92 μL, 0.701 mmol) and stirred at room temperature for 3 days. After that period, the reaction mixture was treated with an additional amount of TMSBr (20 μL) and stirring was continued for 2 more days. The reaction mixture was then treated with MeOH and stirred for 1 hr. The solvent was removed under vacuum, the residue was dissolved in MeOH and triturated with CH₂Cl₂ to give Example 26-1 as white solid (31 mg, 100%). ¹H NMR (500 MHz, D₂O) δ 8.19 (s, 1H), 7.33 (d, J=8.0 Hz, 2H), 7.27 (d, J=8.0 Hz, 2H), 3.49 (s, 3H), 3.43-3.46 (m, 2H), 3.35-3.28 (m, 2H), 2.30 (s, 3H); ¹³C NMR (125 MHz, D₂O) δ 162.62, 159.61, 147.97, 139.35, 137.58, 129.62, 129.56, 129.24, 129.15, 122.69, 53.66, 34.20, 21.97, 20.29; ³¹P NMR (81 MHz, DMSO) 6-9.64.

### Preparation of Example 27-1 from Intermediate 30 (where R6 is Tolyl)

### Step 1

In a flask, a solution of tetraethyl methylenediphosphonate (30 μL, 0.121 mmol) in THF (2 mL) was cooled to 0° C. and 60% NaH (5.8 mg, 0.146 mmol) was added in a portion. The mixture was stirred at 0° C. for 15 min. To this mixture intermediate 30 (38 mg, 0.134 mmol; where R6 is tolyl) in THF (1 mL), was added and stirring was continued at room temperature for 1 hr. The reaction was quenched with MeOH. The solvent was removed under vacuum. The residue was purified by chromatography on silica gel (100% EtOAc to 10% MeOH in EtOAc) to give (E)-diethyl(2-(4-methoxy-6-(p-tolyl)thieno[2,3-d]pyrimidin-5-yl)vinyl)phosphonate as colorless oil (36.3 mg, 65%). ¹H NMR (300 MHz, CDCl₃) δ 8.04 (s, 1H), 7.84 (dd, J=24.2, 17.8 Hz, 1H), 7.33 (d, J=8.0 Hz, 2H), 7.22 (d, J=8.0 Hz, 2H), 6.40 (dd, J=20.4, 17.8 Hz, 1H), 4.05 (m, 4H), 3.59 (s, 3H), 2.37 (s, 3H), 1.28 (t, J=7.1 Hz, 6H); ¹³C NMR (75 MHz, CDCl₃) δ 163.15, 157.94, 147.05, 142.04, 139.90 (d, J=7.9 Hz), 139.32, 129.75, 129.71, 129.52, 128.64 (d, J=26.2 Hz), 122.19, 121.13, 118.68, 61.78 (d, J=5.4 Hz), 34.36, 21.29, 16.34 (d, J=6.6 Hz); ³¹P NMR (81 MHz, CDCl₃) δ 19.12.

### Step 2

The above alkene (36 mg, 0.086 mmol) was dissolved in EtOH (3 mL) and a catalytic amount of Pd/C (20 mg) was added. The reaction mixture was stirred under an atmosphere of H₂ for 2 days. The solution was filtered through celite and washed with MeOH. The filtrate was concentrated to give the hydrogenated product diethyl(2-(4-methoxy-6-(p-tolyl)thieno[2,3-d]pyrimidin-5-yl)ethyl)phosphonate as a white solid (36 mg, 100%). ¹H NMR (300 MHz, CDCl₃) δ 8.02 (s, 1H), 7.32 (d, J=8.1 Hz, 2H), 7.23 (d, J=8.1 Hz, 2H), 4.06 (m, 4H), 3.58 (s, 3H), 3.15-3.24 (m, 2H), 2.38 (s, 3H), 2.31-2.11 (m, 2H), 1.26 (t, J=7.0 Hz, 6H); ¹³C NMR (75 MHz, CDCl₃) δ 162.98, 158.07, 146.47, 138.49, 136.81, 132.77, 132.51, 129.73, 129.60, 129.48, 123.08, 61.47 (d, J=6.1 Hz), 34.07, 27.49, 25.68, 21.22, 16.37 (d, J=6.3 Hz); ³¹P NMR (81 MHz, CDCl₃) δ 31.37.

### Step 3

A solution of diethyl(2-(4-methoxy-6-(p-tolyl)thieno[2,3-d]pyrimidin-5-yl)ethyl)phosphonate (36 mg, 0.086 mmol) in CH₂Cl₂ (3 mL) was treated with TMSBr, followed by MeOH as previously described for the hydrolysis of the diethyl ester precursor of Example 26-1 in order to obtain the free mono-phosphonic acid inhibitor of Example 27-1 (i.e. compound 32, where R6 is tolyl; Scheme 6) as white solid (21 mg, 68%). ¹H NMR (500 MHz, D₂O) δ 8.20 (s, 1H), 7.42 (d, J=8.0 Hz, 2H), 7.34 (d, J=8.0 Hz, 2H), 3.52 (s, 3H), 3.05-3.10 (m, 2H), 2.37 (s, 3H), 1.63-1.70 (m, 2H); ¹³C NMR (125 MHz, D₂O) δ 162.73, 159.43, 147.78, 139.06, 135.25, 135.14, 129.79, 129.70, 129.65, 122.70, 34.22, 31.09 (d, J=127.3 Hz), 23.27, 20.32; ³¹P NMR (81 MHz, D₂O) δ 20.42.

Compounds of general structure 35 (Scheme 6) can be prepared from the common intermediate 29 using various experimental procedures known to those skilled in the art of organic synthesis (for examples see Lockma, J. W. et al. Synth Commit 2012, 42, 1715-1723; Mylari, B. L. et al. J. Med. Chem. 2001, 44, 2695-2700; DeLuca L. and Giacomelli, G. J. Org. Chem. 2003, 68, 4999-5001; Schmidt, A.-K. C. and Stark, C. B. W. Org Lett 2011, 13, 4164-4167)

### Preparation of Example with General Structure 38 and 39 from Intermediate 16 (where R6 is Tolyl)

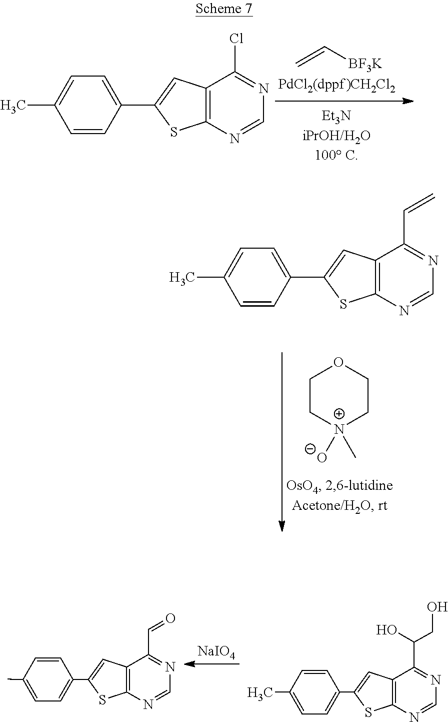

### Step 1

In a vial, a mixture of 4-chloro-6-(p-tolyl)thieno[2,3-d]pyrimidine (i.e. 16; FIG. 7), potassium vinyltrifluoroborate, and PdCl₂(dppf)CH₂Cl₂ was combined and purged with argon. PrOH/H₂O and Et₃N were added and the mixture was purged again with argon. The mixture was heated at 100° C. for 1 hour. The solution was passed through celite, washed with EtOAc, concentrated, and purified by chromatography (100% Hex to 20% EtOAc in Hex) on silica gel to give intermediate 36 (where R6 is tolyl) as yellow solid. The ¹H NMR and MS were consistent with the desired product.

¹H-NMR (CDCl₃): δ 2.40 (s, 3H, —CH₃), 5.85 (d, J=10.70 Hz, 1H, —CH═CH₂), 6.77 (d, J=10.70 Hz, 1H, —CH═CH₂), 7.16-7.23 (m, 1H, —CH═CH₂), 7.26 (d, J=8.10 Hz, 2H, Phenyl), 7.57 (s, 1H, 5-H thienopyrimidine), 7.60 (d, J=8.10 Hz, 2H, Phenyl), 8.97 (s, 1H, 2-H thienopyrimidine).

### Step 2

To a solution of thienopyrimidine-alkene derivative 36 (Scheme 7) in 10:1 acetone:water (4 mL), 2,6-lutidine, 4-methylmorpholine-N-oxide, and osmium tetraoxide (0.1 mL of a 0.0404 M solution in toluene) were added. The mixture was stirred for 2 h at room temperature (at which point LCMS indicated complete conversion of 36 to the desirable diol). Then, 1 mL water was added followed by NaIO₄ in small portions and the mixture was stirred at room temperature for 1 hour. The reaction was quenched with saturated aqueous solution of sodium thiosulfate (10 mL), the mixture is extracted with ethyl acetate (3×15 mL), washed with saturated aqueous solution of ammonium chloride, dried over anhydrous MgSO₄, and concentrated under vacuum. The crude residue was purified by flash column chromatography on silica gel eluted with hexane-ethyl acetate (7:1) to give intermediate 37 (where R6 is tolyl) as a yellow solid.

¹H-NMR (CDCl₃) δ: 2.43 (s, 3H, —CH₃), 7.30 (d, J=8.00 Hz, 2H, Phenyl), 7.70 (d, J=8.10 Hz, 2H, Phenyl), 8.31 (s, 1H, 5-H thienopyrimidine), 9.24 (s, 1H, 2-H thienopyrimidine), 10.25 (s, 1H, —CH═O).

The conversion of intermediate 37 (Scheme 7) to Examples of general structure 38 and 39 was achieved using the same protocol as those previously described for the preparation of inhibitors with general structure 32 and 33 (Scheme 6), such as Example 26-1 and Example 27-1.

### Inhibitors of the Human Farnesyl Pyrophosphate Synthase

In vitro Enzymatic Inhibition Assay for hFPPS:
In vitro sensitized inhibition assay for hFPPS (M2):

All assays were run in triplicate using 4 ng of the human recombinant FPPS (˜1 nM hFPPS) and 0.2 μM of each substrates, GPP and IPP (³H-IPP, 3.33 mCi/mmol) in a final volume of 100 μL buffer containing 50 mM Tris pH 7.7, 1 mM MgCl₂, 0.5 mM TCEP, 20 μg/mL BSA and 0.01% Triton X-100. For assays run with a 10 min pre-incubation period, the enzyme and inhibitor were incubated in the assay buffer in a volume of 80 μL at 37° C. for 10 min. After 10 min, the substrates were added to start the reaction and also bring the inhibitor and substrate to the desired final concentrations. After addition of all substrates, all assays were incubated at 37° C. for 8 min. Assays were terminated by the addition of 200 μL of HCl/methanol (1:4) and incubated for 10 min at 37° C. The assay mixture was then extracted with 700 μL of ligroin (in order to separate reaction products from unused substrate), dried through a plug of anhydrous MgSO₄ and 300 μL of the ligroin phase was combined with 8 mL of scintillation cocktail. The radioactivity was then counted using a Beckman Coulter LS6500 liquid scintillation counter.

TABLE 1
The in vitro potency of select examples are shown below and compared
to the potency of known literature examples of inhibitors of the human
FPPS that were tested in the same assay

| | IC₅₀ (μM) in |
|---|---|
| Compound | hFPPS Method 2 |
| Risedronic acid | 0.005 |
| Inhibitor 1* | 0.92 |
| Inhibitor 2* | 5.7 |
| 5-1 | 0.022 |
| 6-1 | 0.021 |
| 7-1 | 0.036 |
| 8-1 | 0.015 |
| 10-1 | 0.063 |
| 11-1 | 0.014 |
| 13-1 | 0.011 |
| 16-1 | 29 |
| 18-1 | 4.5 |
| 21-1 | 2.7 |
| 26-1 | 50 |
| 33-1 | 40 |
| 36-1 | 4.2 |
| 37-1 | 1.3 |
| 41-1 | 10 |

*Inhibitor 1 and Inhibitor 2 in the table refer to the compounds identified in the background section

### Cell Culture and Viability Assays in Multiple Myeloma Cells:

The RPMI 8226 multiple myeloma cell line was obtained courtesy of Dr. Leif Bergsagel (Mayo Clinic, Scottsdale, Ariz.) and cultured in RPMI-1640 medium supplemented with 10% fetal bovine serum (Gibco BRL, Gaithesburg, Md.) supplemented with 2 mM L-glumatime in a 5% CO₂ atmosphere at 37° C. A dilution method was used to determine EC₅₀ values for inhibition for each target compound; compounds were diluted in culture medium. Cells were seeded in 96 well plates at a density 10,000 cells per well incubated for 2 h before the addition of 10 μL of compound at half-logarithmic dilutions from 100 nM to 333 μM with a fixed final volume. Plates were then incubated for 72 h at 37° C. in the presence 5% CO₂, following which an MTT, 4,5-dimethylthiazole-2-yl)-2,5-diphenyltetrazolium bromide reagent was used according to the manufacturers documentation (Promega, Madison, Wis.). Plates were read at OD490 nM on a Dynex MRX microplate reader (Magellan Biosciences, Chelmsford Mass.). Results were analyzed to obtain dose-response curves and EC₅₀ calculations using GraphPad PRISM version 5 (GraphPad Software, San Diego, Calif.).

Compounds were routinely tested at fixed concentrations of 10 μM and 100 μM in the above described antiproliferation assay using the multiple myeloma cells RPMI 8226. A 20-40% decrease in cell proliferation was observed with many compounds described in this inventions at 100 μM. A dose-dependent inhibition study was performed only with the most potent analogs; an example is shown below and compared to the potency of zoledronic acid and risedronic acid

| | EC₅₀ (μM) in MM cells |
|---|---|
| Compound | RPMI-8226 |
| Zoledronic acid | 11 |
| Risedronic acid | 13 |
| Example 7-1 | 8.5 |

### Phospho-Tau Bioassay:

The commercially available INNOTEST® PHOSPHO-TAU(181P) solid-phase enzyme immunoassay was used. In this assay, the phosphorylated Tau protein or fragments are captured by a first monoclonal human specific antibody, HT7 (IgG1). Human immortalized neurons were treated with various compounds at Human cell culture homogenates are added and incubated with biotinylated AT270 (IgG1) monoclonal. This antigen-antibody complex is then detected by a peroxidase-labeled streptavidin. After addition of substrate working solution, samples develop a color. The color intensity is a measure for the amount of phosphorylated Tau protein in the sample. This assay has been standardized in numerous research laboratories (for examples see Vanderstichele, H. et al. Alzheimer's & Dementia 2012, 8, 65-73; Zimmermann, R. et al. Journal of Alzheimer's Diseases 2011, 25, 739-745; Blennow, K. et al. Molecular and Chemical Neuropathology 1995, 26, 231-245). Human neuroblastomas SH-SY5Y cell line were used and purchased at the ATCC under the label CRL2266. All compounds were tested in three fixed concentrations at 100 nM, 1 μM and 100 μM in duplicates. We used the 6 well culture plates to grown a significant density of neurons (330,000 cells/well). Cells were grown to 80% confluence in MEM-F12 media with 10% fetal bovine serum, the exposed for 24 hours to the different inhibitors. After one day, the supernatant was removed (and saved at −20° C.). The cell are washed repeatedly in PBS buffer and saved at −80° C. ELISA assays were performed on neuronal cell homogenates using a standardized assay developed by INNOGENETICS called Innotest Total Tau also refers to as the hTau assay and Innotest phospho-Tau (P181). Assays were performed as per manufacturer protocol. The concentration units are in pg of P-Tau per μg of total protein in the cell homogenate. However, the effects of zoledronic acid and risedronic acid on the levels of P-Tau and T-Tau cannot not be properly evaluated due to the fact that these compounds are highly toxic; compounds which cause stress or damage to the neurons stimulate the production of P-Tau protein. Lactate dehydrogenase (LDH) activity was measured using a commercial kit; the maximum toxicity was based on % lactic acid dehydrogenases activity in the medium; the control was set at zero.

TABLE 2
Modulation of total Tau (T-Tau) and phospho-Tau (P-Tau) levels
in human immortalized neurons by hFPPS inhibitors.

| Compound | | | | | |
|---|---|---|---|---|---|
| (100 nM) | P-Tau | T-Tau | Ratio P/T | LDH^{b} | Toxicity |
| control | 0.4 | 34 | 0.012 | 0% | none |
| Zoledronic acid | 0.19 | 34 | 0.006 | 60% | high |
| Risedronic acid | 0.17 | 38 | 0.004 | 60% | high |
| Inhibitor 1* | 0.30 | 65 | 0.004 | 3% | minor |
| Example 11-1 | 0.30 | 31 | 0.007 | 0% | none |
| Example 16-1 | 0.35 | 47 | 0.007 | 7% | minor |

*Inhibitor 1 in the table refer to the compounds identified in the background section

1. A compound of formula I: 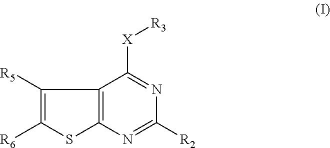
or a pharmaceutically acceptable salt or solvate thereof, wherein
X═O, NR4, or CR4R4;
R2 is selected from H, C1-6alkyl, C3-6 cycloalkyl, C6-10aryl, 3-10 membered heterocycle, —CONHR7, —SO2NHR7;
R3 is selected from CH[PO(OH)2]2; CH2PO(OH)2; CHR7PO(OH)2; CH(CO2H)2; CH(SO2NHR7)PO(OH)2; CR8R9-SO2NR7(PO(OH)2), COCO2H; CR8(PO(OH)2)2, CR8R9CO2H; CR8R9PO(OH)2, CR8R9COR10 or C1-6alkyl;
R4 are each independently H, C1-6alkyl, aryl or 3-10 membered heterocycle;
R5 and R6 are independently selected from H, C1-6alkyl, optionally substituted C3-6 cycloalkyl, optionally substituted C6-10aryl, optionally substituted 3-10 membered heterocycle, CH2OH, CO2H, CH2CO2H, (CH2)nPO(OH)2, (CH2)n-SO2NR7(PO(OH)2), (CH2)nSO2NR7R8, NR7R8, NH(CH2), PO(OH)2, NO2 or OR7; where n is an integer number from 1-3;
R5 and R6 can also be independently selected from amino acids, natural or unnatural attached to thienopyrimidine core via a C-1-4 alkyl linker;
R7, R8 and R9 are each independently —H, —C1-6 alkyl, —C3-6 cycloalkyl, —C6-10 aryl, 3-10 membered heterocycle or —C1-6alkyl-C6-10aryl;
R8 and R9 can also be taken together to form a 3 to 6 membered cycoalkyl; and
R10 is C1-6 alkyl, —C3-6 cycloalkyl, —C6-10 aryl or 3-10 membered heterocycle.

2. The compound of claim 1, or a pharmaceutically acceptable salt or solvate thereof wherein R3 is selected from CR8[PO(OH)₂]₂, CR8R9CO₂H; CR8R9PO(OH)₂ or CR8R9-SO2NR7(PO(OH)2).

3. The compound of claim 1, or a pharmaceutically acceptable salt or solvate thereof wherein X is NR4.

4. The compound of claim 1, or a pharmaceutically acceptable salt or solvate thereof wherein X is NH.

5. The compound of claim 1, or a pharmaceutically acceptable salt or solvate thereof wherein X is CR4R4.

6. The compound of claim 1, or a pharmaceutically acceptable salt or solvate thereof wherein R4 is independently H, C1-6alkyl or aryl.

7. The compound of claim 1, or a pharmaceutically acceptable salt or solvate thereof wherein R5 is selected from H, C1-6alkyl, optionally substituted C3-6 cycloalkyl, optionally substituted C6-10aryl, optionally substituted 3-10 membered heterocycle, CH₂OH, CO₂H, CH₂CO₂H, (CH₂)ₙPO(OH)₂, (CH₂)ₙ(SO₂NHR7)PO(OH)₂ (CH₂)ₙSO₂NR7R8, NR7R8, NH(CH₂), PO(OH)₂, or OR7; where n is an integer number from 1-3.

8. The compound of claim 1, or a pharmaceutically acceptable salt or solvate thereof wherein R5 is H; C1-6alkyl, phenyl, CO₂H, CH₂CO₂H, CH₂PO(OH)₂, NO₂, NR7R8, or OR7.

9. The compound of claim 1, or a pharmaceutically acceptable salt or solvate thereof wherein R6 is independently selected from optionally substituted C3-6 cycloalkyl, substituted phenyl, optionally substituted naphtyl, optionally substituted 3-10 membered heterocycle.

10. The compound of claim 1, or a pharmaceutically acceptable salt or solvate thereof wherein R6 is independently selected from optionally substituted C3-6 cycloalkyl, optionally substituted phenyl, optionally substituted naphthyl, and optionally substituted 3-10 membered heterocycle.

11. The compound of claim 1, or a pharmaceutically acceptable salt or solvate thereof wherein R2 is H.

12. A pharmaceutical composition comprising a compound as defined in claim 1 or a pharmaceutically acceptable salt or solvate thereof, and an acceptable excipient.

13. (canceled)

14. A method for treating or preventing osteoporosis, cancer,
lowering of cholesterol, neurodegenerative diseases, bacterial infection, viral infection, infection with protozoa, Alzheimer's disease, related disorders, or tauopathies comprising administering to a subject in need thereof a therapeutically effective amount of a compound as defined in claim 1, or a pharmaceutically acceptable salt or solvate thereof.

15-21. (canceled)

22. The method of claim 14 for treating or preventing osteoporosis, cancer, lowering of cholesterol, neurodegenerative diseases, bacterial infection, viral infection, or infection with protozoa.

23. The method of claim 14 for treating or preventing Alzheimer's disease, related disorders, or tauopathies.
